(19) United States
(12) Patent Application Publication
(10) Pub. No.: US 2025/0114317 A1
(43) Pub. Date: Apr. 10, 2025

(54) Use of L-cysteine for improving the structure and function of gastric mucosa

(71) Applicant: Biohit Oyj, Helsinki (FI)

(72) Inventor: Osmo Suovaniemi, Helsinki (FI)

(21) Appl. No.: 18/725,842

(22) PCT Filed: Dec. 27, 2022
(86) PCT No.: PCT/FI2022/050872

§ 371 Date: Jul. 1, 2024

(30) Foreign Application Priority Data
Dec. 29, 2021 (FI) 20216361

(51) Int. Cl. A61K 31/198; A61K 9/00; A61K 9/48; A61P 1/04; G01N 33/68

(52) U.S. Cl. CPC A61K 31/198; A61K 9/0065; A61K 9/48; A61P 1/04; G01N 33/6893; G01N 2800/062

(57) ABSTRACT

According to an example aspect of the present invention, there is provided an acetaldehyde-binding preparation for use in a method for improving the structure and physiological function of gastric mucosa among subjects with Helicobacter pylori-associated or autoimmune type atrophic gastritis, or with long-term use of PPIs.

### FIELD

The present invention relates to an acetaldehyde-binding preparation for use in a method for improving, restoring and/or recovering the structure and function of gastric mucosa among subjects with acid-free stomach and/or Helicobacter pylori-associated (HP+) or autoimmune type (HP−) atrophic gastritis, with a natural amino acid, slow-release L-cysteine (Acetium® capsules, Biohit Oyj), thereby preventing atrophic gastritis and its progression into gastric cancer. GastroPanel® test (Biohit Oyj) is used for initial screening of the subjects and for demonstrating the improved function of the atrophic corpus mucosa after a valid period of L-cysteine administration.

### BACKGROUND

Atrophic gastritis (AG) is a disease associated with a significantly increased risk of gastric cancer, being the single most important precursor condition for gastric cancer (GC) known so far. On the other hand, H. pylori (HP)-infection is the most important causative agent in the development of gastritis, and subsequent AG. HP-infection and AG in the antrum may lead to GC and peptic ulcer disease. It is well known that a minority of AG cases in the corpus develop by autoimmune mechanisms. The risk of GC is 4-5 times higher among patients suffering from severe atrophy of the corpus (AGC) mucosa as compared with their healthy counterparts. Among the patients with severe atrophy in the antrum (AGA), this risk is 18-fold higher than in healthy subjects, and the risk increases up to 90-fold, if severe atrophy affects both antrum and corpus (i.e., with severe pan-atrophy, AGP).

The prevalence of AG and GC increases with increasing age, and the risk for both diseases is highest among the subjects >45 years of age. The majority of GCs among the elderly are of the intestinal subtype, developing through the AG-to-GC sequence (Correa cascade). Because of the high GC risk among the elderly, the current consensus recommendations suggest endoscopy for all dyspeptic elderly people as well as for those aging above 45 (50) years.

Based on extensive documentation by a large number of cohort studies conducted during the past several decades, a relatively clear picture has emerged concerning the stepwise development of invasive GC.¹⁻²⁴ This increased understanding of the pathogenesis of GC has also prompted the scientific community to develop international recommendations for the management of the precancerous conditions of the stomach.²⁵ These management guidelines are based on three key statements, of which a relatively good consensus prevails among the leading gastroenterology experts in Europe.²⁵

1) Patients with chronic atrophic gastritis (AG) or intestinal metaplasia (IM) are considered to be at higher risk for gastric adenocarcinoma; 2) High grade dysplasia and invasive carcinoma should be regarded as preventable outcomes, when the patients with chronic AG or IM are adequately managed; and 3) Patients with endoscopically detectable high grade dysplasia or carcinoma should undergo staging and adequate management.²⁵

In most instances, the development of so-called “intestinal” GC represents the culmination of an inflammation-metaplasia-dysplasia-carcinoma sequence, known as the Correa cascade of multistep gastric carcinogenesis,^{16,19-21} where disease progression occurs from a normal mucosa through chronic non-atrophic gastritis, AG, and IM, to dysplasia, and finally to GC, as consistently confirmed in several studies.^{26,27}

Gastric mucosal atrophy (AG) and IM confer a high risk for the development of GC as they constitute the precursors where dysplasia and intestinal-type gastric adenocarcinoma develop.²⁸⁻³¹ Thus, chronic AG and IM are considered as cancer precursor lesions.²⁵ Chronic AG is diagnosed and graded on the basis of the presence of chronic inflammatory cells, including lymphocytes and plasma cells that infiltrate the connective tissue stroma, associated with the disappearance of the normal glands.^{8,14,32-34} The severity of gland loss (atrophy) should be graded, as mild, moderate or severe, albeit the inter-and intra-observer agreement is not satisfactory.²⁵

Individuals may develop different phenotypes of chronic gastritis due to different genetic profiles and environmental exposure. Cases of inflammatory changes limited to the antrum and without gland atrophy and/or IM are defined as diffuse antrum gastritis.^{8,14} In contrast, cases with gland atrophy and/or IM distributed multi-focally (including the lesser curvature of the corpus and fundus), are best defined as multifocal AG. When associated with AG of the antrum, the condition is called atrophic pan-gastritis (AGP).⁸

The mucosal changes in the stomach should be described in terms of the i) severity and ii) distribution of any premalignant conditions/lesions. Several classification schemes have been developed for chronic gastritis and pre-neoplastic changes. At present, the updated Sydney System (USS) is widely used both in clinical practice and in research, combining topographic, morphological, and etiological information in reporting systems designed to include both grading and staging of gastritis.^{35,36} More recently, the systems known as OLGA (operative link for gastritis assessment), and OLGIM (operative link on gastric intestinal metaplasia) assessment have been proposed for staging of gastritis.³⁷ Meritorious as they are, both classifications suffer from an unsatisfactory inter-and intra-observer reproducibility.

Gastric dysplasia represents the penultimate stage in gastric carcinogenesis,^{16,19-21,38} defined as histologically unequivocal neoplastic epithelium without evidence of tissue invasion, i.e., a full-blown neoplastic precancer lesion. It is characterized by cellular atypia reflective of abnormal differentiation, and disorganized glandular architecture.³⁹⁻⁴¹ Both the correct diagnosis and grading of dysplasia are critical, because they predict both the risk of malignant transformation and the risk of metachronous GC. The reported progression rates of dysplasia to GC vary within a wide range, from 0% to 73% per year.^{8,14,25,42,43} Undoubtedly, these wide variations reflect the diverse factors, including differences in study design and populations under study and also differences in definitions and assessment of gastric dysplasia.²

There are well-known differences between Japanese and European/North American pathologists in categorizing gastric dysplasia.^{25,35,36} While in Japan, non-invasive intra-mucosal neoplastic lesions with high grade cellular and architectural atypia are termed “non-invasive intra-mucosal carcinoma,” the same lesions are diagnosed as “high grade dysplasia” by most pathologists in the West.^{44,45} In an attempt to resolve this issue, the World Health Organization (WHO) recently launched its new classification of gastric precancer lesions.⁴⁶ In this new WHO classification of dysplasia/intraepithelial neoplasia, the widespread use of both dysplasia and intraepithelial neoplasia (IEN) is acknowledged, and these terms are used as synonymous. According to the current WHO classification, the following diagnostic categories should be recognized:
1. Negative for intraepithelial neoplasia/dysplasia
2. Indefinite for intraepithelial neoplasia/dysplasia
3. Low-grade intraepithelial neoplasia/dysplasia
4. High-grade intraepithelial neoplasia/dysplasia
5. Intra-mucosal invasive neoplasia/intra-mucosal carcinoma

In WHO classification, category 1 (negative for intraepithelial neoplasia/dysplasia), includes two important precancerous conditions (chronic AG and IM) (see 1.1.1). Whenever there is any doubt, whether a lesion is neoplastic or non-neoplastic, particularly in small biopsies exhibiting inflammation, the WHO category 2(indefinite for intraepithelial neoplasia/dysplasia) should be used. In such cases, the issue can be usually solved by cutting more sections, by obtaining additional biopsies, or after controlling for possible etiologies.²⁵

Intraepithelial neoplasia/dysplasia comprises unequivocally neoplastic epithelial proliferations, characterized by variable cellular and architectural atypia, but without convincing evidence of invasion. Low-grade intraepithelial neoplasia/dysplasia shows minimal architectural disarray and only mild-to-moderate cytological atypia. In contrast, high-grade intraepithelial neoplasia/dysplasia encompasses neoplastic cells that depict a high nuclear/cytoplasm ratio, prominent nucleoli, more pronounced architectural disarray, and numerous mitoses, many of which are atypical. As repeatedly stated, most patients with high-grade dysplasia lesions are at high risk for either synchronous invasive GC or its rapid development.^{8,14,25,42,43}

Intra-mucosal invasive neoplasia/intra-mucosal carcinoma defines carcinomas that invade the lamina propria and are distinguished from intraepithelial neoplasia/dysplasia not only by desmoplastic changes that can be minimal or absent, but also by distinct structural anomalies, such as marked glandular crowding, excessive branching, budding, and fused or cribriforming glands.²⁵ The diagnosis of intra-mucosal carcinoma (also called carcinoma in situ) indicates that there is an increased risk of lymphatic invasion and lymph-node metastasis. However, endoscopic techniques allow treatment without open surgery, particularly for lesions ≤2 cm in size and for those that are well differentiated with no lymphatic invasion.²⁵

It is estimated that 50% of all GC cases develop through the “Correa cascade”,^{16,19,20,21} leading from HP-associated gastritis to mucosal atrophy, IM, dysplasia, to invasive adenocarcinoma. The concept of classifying AG and IM as precancerous conditions of the gastric mucosa is based on well documented data from long-term prospective cohort studies, demonstrating that the risk of GC is significantly increased among patients with AG, subjected to long enough follow-up.^{1-24,28-31} Indeed, based on this convincing evidence, AG is currently considered as the single most powerful independent risk factor for distal (non-cardia) GC.^{8,16,17,18} Because this process takes several decades, there should be good prospects for early detection of precancer lesions,²² but the problem is a lack of a suitable test for GC screening.²³ Furthermore, most of the patients report only a short period of symptoms before the diagnosis of GC, and up to 40% report no dyspeptic symptoms at all.²⁴

In the pathway towards GC,^{16,19-21} the next step following AG (and frequently accompanying it) is the condition known as intestinal metaplasia (IM). Similar as with gastritis, different phenotypes have recently been recognized also for IM. Traditionally, IM has been classified as “complete” or “incomplete.” Complete intestinal metaplasia (“small-intestinal” or type I) displays goblet and absorptive cells, decreased expression of gastric mucins (MUC1, MUC5AC, and MUC6), and expression of MUC2 (an intestinal mucin). Incomplete intestinal metaplasia (“enterocolic” or type IIA/II, and “colonic” or type IIB/III), displays goblet and columnar non-absorptive cells, in which gastric mucins (MUC1, MUC5AC, and MUC6) are co-expressed with MUC2.

Recently, another pattern of metaplasia, termed spasmolytic polypeptide-expressing metaplasia (SPEM), has been described.⁴⁷ This is characterized by the expression of the TFF2 spasmolytic polypeptide that is associated with oxyntic atrophy. SPEM, which characteristically develops in the gastric body and fundus, appears to share some characteristics with pseudopyloric metaplasia, has a strong association with chronic HP-infection and gastric adenocarcinoma, and may represent another pathway to gastric neoplasia. At present, however, identification of SPEM is not yet among the diagnostic routine of gastric biopsies.

There is no disagreement that biopsy specimens of the stomach are essential in diagnosis and grading of gastric precancer lesions.^{25,49b} The updated Sydney System (USS) is the most widely accepted classification and grading of gastritis.^{35,36} The system was primarily designed to provide standardization for reporting of gastric biopsies. The updated version recommended five biopsies, two from the antrum (3 cm from the pylorus, greater and lesser curvatures), one from the incisura, and two from the corpus (one from the lesser curvature, 4 cm proximal to the incisura, and one from the middle of the greater curvature). Although this biopsy protocol generally correctly establishes HP-status and chronic gastritis, the number of biopsies is controversial with regard to adequate staging of premalignant gastric lesions, mainly because of the multifocal nature of these lesions.^{48,49,49b} This multifocal nature affects their detectability, in turn affecting decisions regarding the patient's therapy or future surveillance.^{19,21}

To obviate the excessive use of this invasive and expensive procedure (endoscopy), there has been an urgent need to develop non-invasive diagnostic tools capable of accurately detecting the patients at high risk for GC, i.e., the different phenotypes of gastritis as well as their related HP-infections.^{50,51} For this purpose, a Finnish biotechnology company (Biohit Oyj) launched an ELISA-based assay designed to measure the concentrations of four stomach-specific biomarkers from a single blood sample. The test is known as GastroPanel®, combining serum pepsinogen I (PGI) and II (PGII), gastrin-17 (G-17) and HP IgG antibodies (IgG-HP). This test is proposed as the first-line diagnostic test for dyspeptic symptoms,⁵²⁻⁵⁴ capable of accurately diagnosing the phenotypes of gastritis, including AG in both the corpus (AGC) and antrum (AGA). Thus, GastroPanel® examination is the first non-invasive diagnostic tool providing possibilities for detecting the patients at risk for GC and peptic ulcer diseases as well as the AG-accompanying conditions (malabsorption of vitamin B12, iron, magnesium, calcium and some drugs).⁵⁴ According to a recent meta-analysis, serum PGs are not suitable for GC screening, however, but they proved to be useful for detecting the patients at risk for GC.⁵⁵ Consequently, these stomach-specific biomarkers are recommended by international experts for diagnosis and screening of AG.⁵⁶

Whether gastric carcinogenesis progressing through the stepwise manner from HP-infection, through AG and IM to invasive GC, once ongoing, can be arrested or even reverted, is a contradictory subject.⁷ This issue is of crucial importance from the clinical point of view, because according to our current thinking, both AG and IM are irreversible conditions once established, and without adequate monitoring, inevitably lead to invasive GC.^{16,19-21}

However, there are some recent implications that an early eradication of HP-infection can slow down or even revert this cascade,^{7,13} suggesting that HP eradication has the potential to prevent gastric cancers.⁵⁷ In a recent study on the effect of HP eradication on patients with premalignant lesions, it was shown that eradication may prevent their progression.⁵⁸ It is thought that a so-called ‘point of no return’ may exist in the histological cascade from chronic gastritis to adenocarcinoma after which eradication is unlikely to prevent GC.⁷ It appears that once IM has become established, eradication, although retarding the progression of IM, cannot completely prevent gastric cancer.^{59,60} This is not necessarily true for gastric atrophy, however, for which there appears to be a discrepancy between the effect of eradication in the corpus and in the antrum. Thus, according to a recent meta-analysis of 12 studies comprising 2.658 patients, eradication of HP-infection results in significant improvement in AGC but not in AGA, and importantly, has no effect on IM of the gastric mucosa.⁶¹ In addition, we do not know whether differences in this respect exist between AG of autoimmune origin and AG due to HP-infection. The former is frequently associated with other autoimmune disorders, including diabetes type 1, and autoimmune thyreoiditis.⁷³

Given the above mentioned considerations, the recent two reports from an Italian group represent a fundamentally novel idea, while suggesting that the function of the gastric mucosa among (HP eradicated) AG patients could be recovered by a simple treatment with a natural (semi-essential) amino acid, L-cysteine (Acetium® capsules, Biohit Oyj, Helsinki, Finland).^{62,63} In their two separate reports, these authors used the GastroPanel® test to demonstrate a markedly improved function of the gastric mucosa during the follow-up of AG patients after treatment with Acetium® capsules.

Thus, these authors assessed the alteration in gastric function after HP-eradication on moderate-severe body atrophic gastritis by determination of sPGI levels and Gastrin 17 (sG 17). A series of 74 dyspeptic patients, selected from 738 consecutive HP-positive patients, with histological features of moderate-severe AG of the corpus, underwent an upper gastrointestinal endoscopy with gastric biopsies and sPGI and sG-17 determination at baseline. All HP-associated AG patients underwent HP-eradication therapy, and serum sPGI and sG-17 were measured again after 6 months, and at 1, 2, 3, 5 and 6 yrs after eradication therapy. In the first report, they had data on 5 patients (4 female, mean age 56, range 49-66 yrs) out of the 74 AG patients, who underwent a 3-month treatment with Acetium® 100 mg (3 capsules daily) before having a meal. In these subjects, the mean levels of sPGI increased from 5.3 μg/L at baseline to 7.8 μg/L after 90 days of Acetium® intake, meanwhile sG-17 remained unchanged (74.6 and 79.8 pmol/L).⁶² Authors concluded that after HP-eradication, these subjects AG of the corpus showed long-lasting improvement of physiological gastric functions, reflected by significantly and stabile increasing of sPGI levels.

In their second report, they had similar 3-month follow-up data from 21/74 of these AG patients (including both autoimmune and HP-related AG), after 3 months of treatment with Acetium® capsules, but 6-year follow-up data for all 74 subjects having undergone HP-eradication.⁶³ Mean sPGI levels prior to HP-eradication were 13.4 μg/L, but increased already at 6 months to 16.6 μg/L (p=0.05), and further to 27.3 μg/L at the completion of the study 6 years later (p=0.01). Conversely, the sG-17 dropped from 84.8 at baseline to 67.6 pmol/L after the 6-year follow up period (p<0.01). Among the 21 patients who received treatment with Acetium®, the mean levels of sPGI increased from 7.9 μg/L at baseline to 11.4 μg/L after 90 days of Acetium® intake, with concomitant drop of sG-17 from 30.3 to 25.5 pmol/L.⁶³ These data clearly implicate that HP-eradication among patients with AG of the corpus results in a long-lasting improvement of the gastric mucosal functions as measured by the GastroPanel® test, with significant and stable increase of sPGI levels and a parallel decreased of sG-17 over a 6-year follow-up period.^{62,63}

Unfortunately, the study design has some inherent weaknesses that preclude making reliable conclusions about the true effect of L-cysteine (Acetium® capsules) in the recovery of gastric mucosal function. Most importantly, the failure to have a biopsy confirmation of the gastric mucosal structure after the 3-month Acetium® capsule intervention (or at study conclusion), makes it impossible to determine, where this apparent normalization of the serum levels of these stomach-specific biomarkers⁵²⁻⁵⁴ is, indeed, associated with concomitant recovery of the AG as well. Similarly, because of the lack of placebo control, there are no means to rule out the possibility that the observed effect is due to HP-eradication only. Because of major conceptual and clinical importance, this concept needs to be assessed in a flawless study design, however.

Acetium® capsules (and lozenges) were designed for inactivation of acetaldehyde in the stomach and in the saliva after alcohol intake and smoking, respectively. This novel concept on the potential therapeutic effect of regular Acetium® capsule intake on both the structure and function of gastric mucosa among subjects with AG, by eliminating acetaldehyde in the stomach contents, would be a definite direct proof of the concept that acetaldehyde is causally associated with the development of GC.

Cysteine is a non-essential amino acid, which was shown (almost 40 years ago) to be capable of eliminating the toxicity of acetaldehyde by reacting covalently with it to form a stable 2-methylthiazolidine-4-carboxylic acid (MTCA).⁶⁴ MTCA is an inert and non-toxic compound that is eliminated from the body through feces and urine, without being absorbed into the blood circulation. This simple principle was used in the recent innovation of Biohit Oyj's Acetium® capsule, which contains 100 mg of L-cysteine.

In the proof-of-concept study, oral administration of Acetium® was confirmed to effectively bind acetaldehyde originated from ethanol metabolism in achlorhydric stomach.⁶⁵ In that setting, the mean acetaldehyde level of gastric juice was 2.6 times higher with placebo than with Acetium® (13 vs. 4.7 μM, p<0.05), implicating that Acetium® can be used to decrease acetaldehyde concentration in non-acidic stomach during alcohol exposure.

This led the authors to examine the concept, whether it would be possible to eliminate alcohol-derived acetaldehyde also from the saliva, using L-cysteine slowly released from a special buccal (Acetium®) tablet.⁶⁶ Indeed, this was shown to be the case in volunteers, in whom, up to two-thirds of acetaldehyde (after alcohol intake) could be removed from the saliva with a slow-releasing buccal L-cysteine formulation (Acetium®). This might have important implications e.g. in prevention of upper GI-tract cancers among individuals with high acetaldehyde exposure (heavy drinkers, smokers).^{66,67,676,67c}

Tobacco smoke contains several classes of carcinogens that include among others polycyclic aromatic hydrocarbons, aromatic amines, and nitrosamines. Tobacco smoke contains also high concentrations of toxic aldehydes,⁶⁸ of which the most abundant is acetaldehyde, its concentrations in tobacco smoke being >1,000 times greater than those of polycyclic aromatic hydrocarbons and tobacco-specific nitrosamines.⁶⁹ Acetaldehyde is also the first metabolite of ethanol oxidation. It binds to DNA, forming stable DNA adducts that are observed in alcohol consumers. Numerous epidemiological studies among alcohol drinkers who have alcohol dehydrogenase (ADH2) deficiency or low aldehyde dehydrogenase (ALDH1B) activity provide the most compelling evidence for the carcinogenicity of acetaldehyde.⁷⁰ This deficiency results in accumulation of acetaldehyde locally in the saliva during ethanol metabolism and is a markedly increased risk for many upper gastrointestinal tract cancers.

Similarly, it was recently shown that acetaldehyde from the tobacco smoke is easily dissolved into the saliva during smoking.⁷¹ Thus, toxic aldehydes could mediate the carcinogenic effect of tobacco smoke through saliva to oral cavity and further down to the larynx, esophagus, and stomach. Based on firm epidemiological and toxicological documentation, IARC proclaimed (in 2009) acetaldehyde as Group I carcinogen, equivalent to asbestos, formaldehyde and others.⁷²

Acetium® capsule is classified as a medical device, and is not a medicine, because of its inherent design; L-cysteine in the capsule is (slowly) released with regulated speed inside the stomach, free L-cysteine reacts with acetaldehyde in the gastric contents, and is not absorbed from the duodenum. This makes the crucial difference to L-cysteine obtained from the foodstuffs as a natural (semi-essential) amino acid, which is liberated only in the duodenum (by pancreatic enzymes) and readily absorbed into blood circulation. This precludes the possibility of Acetium® capsules having any systemic effects, which is a prerequisite to be classified as a medicine.

At the same time, this fact also restricts the framework of the new hypothesis to be tested, while excluding the assumptions that the eventual therapeutic effect of Acetium® capsules on atrophic stomach mucosa and restoration of its functionality could be mediated by some systemic effects. This leaves us less room for building up complex pathways on these mechanisms. Instead, we assume that the therapeutic effects (if established) of Acetium® capsules on atrophic stomach mucosa must be entirely local, in other words mediated by the established efficacy of L-cysteine (Acetium®) to inactivate free acetaldehyde in the stomach, irrespective of its origin (alcohol, smoking or foodstuffs). Given this would necessitate that continuous exposure to acetaldehyde of the gastric mucosa is a prerequisite for sustaining the so called “Correa cascade” in the stomach,^{16,19-21,38,58} Once initiated by the infection of HP, this cascade would continue through the progressing steps of AG and IM to dysplasia (IEN) and GC, unless interrupted by close monitoring of the precursor lesions. To assess whether differences in this respect exist between autoimmune AG and HP-associated AG, both cases will be included in the cohort.

Apart from the studies that confirmed the utility of GastroPanel® in the diagnosis of dyspeptic symptoms and in screening of asymptomatic subjects for the risk conditions of GC, GastroPanel® has been recently studied also in longitudinal settings to assess the biomarker profiles as long-term predictors of GC.^{74,75}

The first of these studies was a matched case-control setting nested within a cohort of Caucasian population in Western Siberia.⁷⁴ Both the cases (GC) and controls (CO) were derived from a population-based cohort (n=9.360) known as HAPIEE (Health, Alcohol and Psychosocial Factors In Eastern Europe) study, enrolled in Novosibirsk (Siberia) during 2003-2005. Altogether, 156 (52 GCs and 104 COs) serum samples collected at base-line were available for GastroPanel® analysis. The biomarker levels below cut-off predicted the development of GC as follows: PGI (2.9; 95% CI: 1.3-6.4), PGII (9.0; 95% CI: 1.8-44.3), PGI/PGII (3.3; 95% CI: 1.5-7.3); G-17 (1.8; 95% CI: 0.7-4.8), and Hp IgG Ab (0.4; 95% CI: 0.1-1.3). In a multivariate model adjusted for sex, age, and all four biomarkers, PGI/PGII ratio was the single most powerful independent predictor of GC (OR=2.9; 95% CI: 1.01-8.0). This was the first time in a Caucasian population, where PGI, PGII and PGI/PGII ratio were shown to be reliable longitudinal predictors of incident GC.⁷⁴

The second longitudinal study was completed in Northern China.⁷⁵ Among 12,112 participants with prospective follow-up from an ongoing population-based screening program, the authors conducted a follow-up, with prediction modeling. In the follow-up, low PGI levels and PGI/II ratios were associated with higher risk of developing GC, similar as low (<0.5 pmol/l) and high (>4.7 pmol/l) G-17 levels [95]. Higher serological biopsy scores based on the five biomarkers at enrollment were associated with higher risk of developing GC during follow-up (p for trend <0.001). According to the authors, Gastro-Panel® test could be used to identify the high-risk individuals for further diagnostic gastroscopy, thus guiding a targeted screening and tailored prevention.⁷⁵

Together with the published meta-analyses,^{76,77} these two studies implicate that due to its high specificity for atrophic gastritis and due to the longitudinal predictive value of individual biomarker profiles, GastroPanel® is truly a test for stomach health and disease. Testing GastroPanel®-negative at any time point during one's life-time precludes (with >95% probability) a significant gastric pathology for several years ahead.^{74,75} In contrast, a GastroPanel® biomarker profile implicating AGC is an independent predictor of an increased risk of contracting GC within the coming 10 years or so.

### SUMMARY OF THE INVENTION

The invention is defined by the features of the independent claims. Some specific embodiments are defined in the dependent claims.

According to an aspect of the present invention, there is provided an acetaldehyde-binding preparation for use in a method for improving the structure and physiological function of gastric mucosa among subjects with acid-free stomach and/or Helicobacter pylori-associated (HP+) or autoimmune type (HP−) atrophic gastritis.

This and other aspects, together with the advantages thereof over known solutions are achieved by the present invention, as hereinafter described and claimed.

The acetaldehyde-binding preparation of the present invention is mainly characterized by what is stated in the characterizing part of claim 1.

Considerable advantages are obtained by means of the invention. For example, the present invention provides that L-cysteine administered by daily intake of Acetium® capsules is an effective remedy to improve the function of the gastric mucosa among patients suffering from atrophic gastritis and to induce concomitant recovery of the atrophic mucosa. Furthermore, the improvement and recovery is achieved without the traditional intervention by any medical treatment or other clinical measures.

Next, the present technology will be described more closely with reference to certain embodiments.

### EMBODIMENTS

The novel technology behind the present invention is simply the following: prolonged L-cysteine administered by daily intake of Acetium® capsules (3×200 mg/d) is an effective remedy to i) improve the function of the gastric mucosa among AG patients who have undergone eradication of HP-infection, and to ii) induce concomitant recovery (reversal) of atrophic gastric mucosa in these subjects.

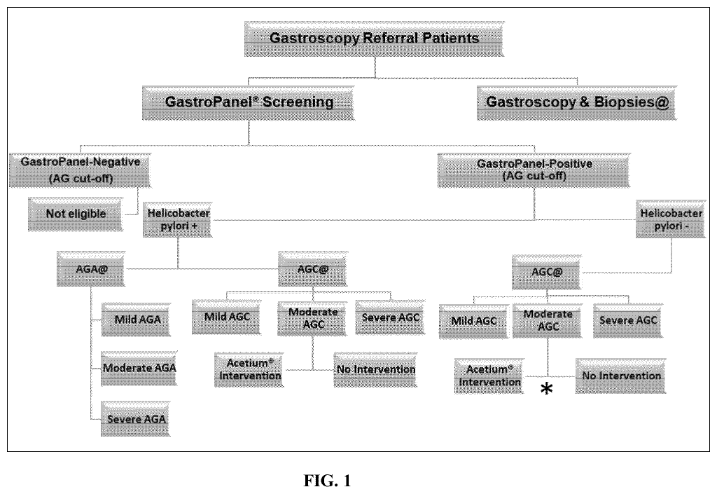

FIG. 1 is a flowchart of the patient enrolment in a cohort study. In the flowchart the “@” grade of AGA and AGC is confirmed by gastroscopic biopsies 1) at baseline and 2) at study conclusion. “*” randomization is performed among subjects with AGC progressed to moderate/severe during the first 3 years of follow-up. Intervention is continued until study conclusion at 60 months.

An essential aspect of the present invention is an acetaldehyde-binding preparation for use in a method for improving the structure and physiological function of gastric mucosa among subjects with acid-free stomach (for whatever reason, such as for example long-term use of PPIs) and/or Helicobacter pylori-associated (HP+) or autoimmune type (HP−) atrophic gastritis, the method comprising at least the following steps:
screening or examining a symptomless subject or a subject having symptoms indicating an autoimmune disease, dyspepsia or reflux symptoms, by quantitatively measuring concentration(s) of a) pepsinogen I (PGI), pepsinogen II (PGII), gastrin-17 (G-17) and Helicobacter pylori antibody (HpAb) biomarkers, or b) pepsinogen I (PGI) biomarker, or c) PGI biomarker, PGII biomarker and PGI/PGII biomarker ratio, or d) G-17 biomarker, from a blood, serum or plasma sample obtained from said subject, and
comparing obtained value(s) to a pre-determined reference range(s) and/or cut-off value(s),
   by administering 50-500 mg of L-cysteine containing slow-releasing preparation according to a pre-determined dosage regime to subjects indicative of acid-free stomach and/or atrophic gastritis based on said measurement and comparison.

According to one embodiment of the present invention, the value(s) is/are indicative for atrophic gastritis, if the PGI concentration in said sample is close to the lower limit or below the reference range or cut-off value, PGI/PGII ratio is close to the lower limit or below the reference range or cut-off value and G-17 concentration is close to the upper limit or above the reference range.

According to a further embodiment of the present invention, the reference range for PGI value is 30-160 μg/l, for PGII 3-20 μg/l, for PGI/PGII ratio 3 or below 3, for G-17S (stimulated) value 5-30 pmol/l, for G-17B (fast) 2-10 pmol/l and the reference range for HpAb 0-30 EIU.

According to an even further embodiment of the present invention, the typical cut-off values for the biomarkers are selected from the group comprising: PGI 30 μg/l, PGI/PGII ratio 3, G-17S value 5 pmol/l, G-17B 2 pmol/l and HpAb 30 EIU.

In one embodiment of the present invention, a long-acting preparation that has a local effect on the stomach” refers to all monolithic or multiparticular tablets or capsules or granules as such, which, when wetted under the influence of the gastric juices adhere to the mucous membrane of the stomach or form a gel that floats in the contents of the stomach, as a consequence of which their residence time in the stomach is prolonged and thus enables a prolonged release in and a local effect of the drug on the stomach. The long-acting preparation that locally acts on the stomach can be a liquid preparation taken orally (mixture), the physical structure of which is a gel.

A special property required of the pharmaceutical composition that has a local effect on the stomach is that it remains in the stomach for as long as possible. Technically, this can be solved in two ways: by making a preparation that adheres to the mucous membrane of the stomach or making a preparation that floats in the contents of the stomach. The preparation can be rendered fixable to the mucous membrane of the stomach by using as additives cationic polymers, such as various chitosan grades. Preparations that float in the stomach are provided by using polymers (e.g., alginic acid) that form a gel and by adding to the preparation sodium hydrogen carbonate, which under the influence of gastric acid releases carbon dioxide, which in turn forms gas bubbles inside the gel. A liquid gel that floats in the stomach can also be prepared from sodium alginate, aluminium hydroxide, sodium hydrogen carbonate, and water, to which the acetaldehyde-binding compound can be added. A corresponding liquid preparation is also obtained by adding an acetaldehyde-binding substance to an aqueous dispersion of chitosan. Another preparation that remains in the stomach for a long time is a preparation, which is known as HBS™ (hydrodynamically balanced system). The preparation can remain in the stomach for a long time, when a relatively large tablet is made of it (with a diameter of at least 7-10 mm) and it is coated with a film, which does not decompose in the alimentary tract, and which, however, releases an effective substance (Oros™) through a hole which has been made to it, for example. However, a prerequisite is that such a preparation be consumed after eating.

A single dose of the pharmaceutical composition having a local effect on the stomach comprises 50-500 mg of acetaldehyde-binding substance; preferably the amount of acetaldehyde-binding substance is 50-300 mg, and most preferably 100-200 mg.

When needed, the dosage is renewed at 4 to 10-hour intervals, preferably at 6 to 8-hour intervals.

The amount of compound released in the conditions of the stomach is preferably 40-80 mg in an hour.

According to one embodiment, the preparation according to the invention, which releases in the stomach, has at least one—often two—polymers, which have the task of keeping the drug as long as possible, for two hours minimum, in the stomach either so that it attaches the preparation to the mucous membrane of the stomach or forms a gel that floats in the contents of the stomach. Another task of the polymers is to prolong the release of the effective substance.

The preparation that locally binds acetaldehyde in the stomach can be a tablet that forms a gel in the stomach or a capsule comprising a mixture of powder or granules that forms a gel. In addition to the acetaldehyde-binding substances, the preparation comprises polymers that form a gel in the stomach, such as chitosans, alginates, sodium carboxy-methylcellulose grades, carbomers or aluminium hydroxide. To advance floating in the stomach, the preparation can also comprise sodium hydrogen carbonate.

The amount of polymers in the preparation is 10-50%, preferably 15-40%, and most preferably 20-30%.

The amount of sodium hydrogen carbonate can be 10-30%, preferably 20% of the amount of polymers.

The preparation that locally binds acetaldehyde in the stomach can be a tablet or granule preparation, wherein the acetaldehyde-binding substance is mixed with the fillers needed and, after that, granulated by using enteric polymers as binders. The binder used can be any known enteric polymer, preferably a polymer with a solution pH of 6-7, and most preferably the polymer is any of the methacrylate derivatives, which are known by the trade names Eudragit L and Eudragit S. The amount of enteric polymer in the preparation is preferably 2-5%, most preferably 3-4%.

The preparation that locally binds acetaldehyde in the stomach can be a liquid preparation, i.e., a mixture comprising, in addition to the acetaldehyde-binding substance, also sodium alginate, aluminium hydroxide, sodium hydrogen carbonate, and water. The amount of water in the whole preparation is 70-90%, most preferably about 75-85%. The amount of sodium alginate in the preparation is preferably 2-10%, most preferably about 5%, and the amount of aluminium hydroxide is preferably 5-15%, most preferably about 10%.

The relative composition of the preparation comprising granules can be as follows, for example:

| | | |
|---|---|---|
| Acetaldehyde-binding substances | 60 | parts |
| Chitosan | 10-40 | parts |
| Calcium hydrogen phosphate | 0-30 | parts |

In one embodiment of the present invention, “A long-acting preparation that has a local effect in the large intestine” refers to all monolithic or multiparticular tablets or capsules or granules as such, which will not release the dose in a prolonged way until the preparation has drifted to the end of the small intestine or all the way to the large intestine.

The preparation according to the invention that releases acetaldehyde-binding substances in the large intestine in a prolonged way, carries the acetaldehyde-binding substance to the last part of the small intestine or to the large intestine before the substance in question is allowed to be released—whichever the releasing mechanism.

The pharmaceutical composition that binds acetaldehyde in the large intestine is administered orally. There are numerous techniques available for directing the release of an orally dosed drug to the large intestine. The most functional solutions are based on the use of enteric polymers. A film coating, which does not dissolve in the acidic environment of the stomach, but dissolves at a pH value of 7 at the latest, can be made both on the tablet and the granules. In making the preparation, it is also possible to use polysaccharides that degrade under the effect of microbes of the large intestine, or polymers generated by azo bonds. The form of preparation known by the trade name Oros™ can also be used, when its opening is first covered with an enteric polymer, the solution pH of which is ≈7.

Useful enteric polymers include, for example, the grades of hydroxypropyl methylcellulose-acetatesuccinate (HPMC-AS) sold by the trade name Aqoat™, Aqoat AS-HF™ in particular, a cellulose acetatephtalate (CAP) grade sold by the trade name Aquateric™, and methacrylic acid-methylmethacrylate copolymers, the grade sold by the trade name Eudragit-S™ in particular.

The preparation according to the invention has at least one ingredient, which adjusts the release of the effective substance not to take place until at the end of the small intestine or in the large intestine. This component can be a polymer that dissolves depending on the pH (=enteric polymer) or a polymer that degrades under the effect of the enzymes secreted by the bacteria of the large intestine. The polymer that controls the place of release can form a film around the entire preparation. It can also form a film around the particles (granules) contained by the multiple-part preparation. The polymer that degrades under the effect of the enzymes secreted by the bacteria of the large intestine can also be as a filler in a monolithic preparation, or as a filler in the granules or in a multiple-unit preparation prepared from these granules.

The preparation according to the invention is an enteric tablet, the film coating of which does not dissolve until at the end of the small intestine or at the beginning of the large intestine. The dissolution pH of the polymer that forms the film is 6.0-7.5, preferably 6.5-7.0. The amount of enteric polymer that forms the film is 5-20%, preferably 10-15% of the whole mass of the tablet. The filler of the tablet can comprise pharmaceutical additives that do not swell, such as calcium hydrogen phosphate.

The preparation according to the invention can also be granules that comprise an acetaldehyde-binding substance and are coated with an enteric film, the dissolution pH of the film-forming polymer being 6.0-7.5, preferably 6.5-7.0. The amount of film-forming enteric polymer of the entire mass of the granule is 5-30%, preferably 15-25%. The granule can comprise 20-40%, preferably about 30% of filler poorly soluble in water, such as calcium hydrogen phosphate.

The binder of the granule coated with the enteric film, according to the invention, can be an enteric polymer, the dissolution pH of which is 6.0-7.5, preferably 6.5-7.0. The amount of binder in the granule is 2-5%, preferably 3-4%.

The preparation according to the invention can also be a tablet comprising the enteric-coated granules described above, on which an enteric film has also been made. The tablet made for such a preparation not only comprises enteric granules, but also a filler suitable for direct compression, such as microcrystalline cellulose, the amount of which in the tablet is 30-70%, preferably 40-60%.

The dosage unit of the pharmaceutical composition preferably comprises 50-500 mg of acetaldehyde-binding substance; preferably the amount of acetaldehyde-binding substance is 50-300 mg, and most preferably 100-200 mg.

The amount of compound releasing in the conditions of the large intestine is preferably 50-100 mg in an hour.

When needed, the dosage can be repeated at 4 to 10-hour intervals, preferably at 6 to 8-hour intervals.

The composition of the enteric tablet, which comprises enteric granules and binds acetaldehyde in the desired way, can be as follows, for example:

| | | | |
|---|---|---|---|
| Enteric granules: | Acetaldehyde-binding substance | 100 | mg |
| | Filler, e.g., calcium hydrogen phosphate | 30-50 | mg |
| | Enteric polymers | 40-60 | mg |
| Enteric tablet: | Enteric granules | 170-210 | mg |
| | Microcrystalline cellulose | 170-210 | mg |
| | Lubricants (e.g. magnesium stearate | | |
| | and talcum) | 5-10 | mg |
| | Enteric polymers | 30-50 | mg |

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### EXAMPLES

### Example 1

The locally long-acting pharmaceutical preparation that binds acetaldehyde in the stomach can be prepared and used to decrease the risk of cancer caused by acetaldehyde as follows:

The relative composition of the preparation that locally binds acetaldehyde in the stomach can be as follows, for example:

| | | |
|---|---|---|
| L-cysteine | 60 | parts |
| Chitosan | 10-40 | parts |
| Calcium hydrogen phosphate | 0-30 | parts |

The powder mixture is mixed by conventional mixers (such as a blender), which are used in the drug industry. After that, the powder mixture is granulated using a 2.5% acetic acid as a granulation liquid. The granulation liquid can be added to the same blender. The moist powder mass is compresses through a screen plate or a perforated plate (the diameter of the aperture being 2 mm). The formed granules are dried and screened. A screen fraction of 1.2-1.7 mm is recovered, which is dispensed into hard gelatine capsules so that the dose of cysteine is 100 mg.

The tablets prepared above are ingested to decrease the risk of cancer locally caused by acetaldehyde in occasions, which are favourable for an increase in the acetaldehyde content of the stomach, such as in connection with consuming alcoholic drinks. The dosage is given at 4 to 6-hour intervals as long as there is alcohol in the blood.

### Example 2

The pharmaceutical composition that releases acetaldehyde-binding substances in the large intestine in a prolonged way can be prepared and used to decrease the risk of cancer caused by acetaldehyde as follows.

The composition of the enteric tablet, which comprises enteric granules and binds acetaldehyde in the desired way, can be as follows, for example:

| | | | |
|---|---|---|---|
| Enteric granules: | L-Cysteine | 100 | mg |
| | Calcium hydrogen phosphate | 40 | mg |
| | Eudragit-S | 5 | mg |
| | Aqoat AS-HF | 40 | mg |
| Enteric tablet: | Enteric granules | 185 | mg |
| | Microcrystalline cellulose | 185 | mg |
| | Magnesium stearate | 4 | mg |
| | Talcum | 4 | mg |
| | Aqoat AS-HF | 40 | mg |

L-cysteine and the calcium hydrogen phosphate that works as a filling agent are mixed together. Eudragit S is dissolved in ethanol (a 20% solution) and the solution is used to moisten the powder mixture. The wet mass is compressed into granules. The dried granules are screened and a granule fraction of 1.2-1.7 mm is coated with Aqoat AS-HF. The composition of the coating solution is as follows: Aqoat AS-HF 10%, triethylcitrate 3.5%, magnesium strearate 3%, and water 83.5%. The coated granules are mixed with microcrystalline cellulose (e.g., Emcocel LP 200™) and, finally, the lubricants are added to the mixture: magnesium stearate and talcum. Next, the mixture is compressed into tablets and, finally, an enteric film is made on the tablet in the same way as on the granules. In all stages of operation, mixers, granulators, screening equipment, film coating equipment, and tablet compressing machines, which are generally used in the pharmaceutical industry, can be used.

The composition prepared above is ingested orally in connection with consuming alcoholic drinks and the dosage is repeated at 4 to 6-hour intervals as long as there is alcohol in the blood.

### Example 3

The present study is designed to validate i) the concept that GastroPanel® biomarker assay is a reliable non-invasive tool in the safe follow-up of the patients with AG, ii) to assess the value of GastroPanel® biomarker profiles as predictors of disease outcome among AG patients during a long-term prospective follow-up, iii) to establish, whether any differences in the clinical outcomes exist between HP+ and HP− patients with AG. In addition, iv) in this longitudinal study, a randomized controlled trial is nested to validate the novel concept that Acetium® capsules effectively interfere with the Correa cascade leading to recovery of stomach functions with concomitant recovery of gastric atrophy (AG), irrespective whether HP-associated or of autoimmune origin.

A longitudinal follow-up study of a cohort of patients with biopsy-confirmed AG at baseline, designed to establish the natural history (clinical outcome) of AG without medical or other intervention. Potentially eligible AG patients are screened among the consecutive gastroscopy-referral patients by GastroPanel® test. Eligible patients are incident cases of AG (in corpus, antrum or both), graded as mild, moderate or severe in degree by the gastroscopy biopsies and the serological biomarker assay. Both the patients with autoimmune AG (HP−) and those testing HP-positive with GastroPanel® test are enrolled. Before enrolment, all subjects are requested to sign a written consent. No medical treatment is instituted to HP-infection, so as not to interfere with the natural disease outcome.

The study is supervised by a steering committee consisting of the members of the Company's Scientific Board and representatives of the partner clinics. These two guidelines give detailed recommendations for patient selection (diagnosis), trial design, as well as evaluation of the results, to be described in the following.

### Aims of the Study

The general goal of the study is to establish the natural history of AG, i.e., the clinical outcome of the disease without intervention by any medical treatment or other clinical measures (e.g. repeated biopsies, mucosal resections, gastric surgery). This natural history of AG is compared with the clinical outcomes in patients undergoing active clinical intervention by regular intake of Acetium® capsules, recently implicated as a potential remedy for AG.

In this longitudinal setting, a cohort of 500 subjects with biopsy-confirmed AG (AGA, AGC, AGP) are being prospectively followed-up by annual monitoring by non-invasive serological biomarker testing (GastroPanel®), repeated at 12-month intervals until completion of a 5 years of follow-up.

During the follow-up, whenever progression of AG is disclosed by GastroPanel® testing, the subjects are being randomised to i) Placebo arm, and ii) Acetium® intervention arm. In the former, the subjects continue the study protocol as before, with no medical intervention, by the 12-month GastroPanel® monitoring, until the end of the follow-up. In the Acetium® arm, the subjects start using Acetium® capsules (3×200 mg on occasion of their daily meals). Otherwise, they continue the GastroPanel monitoring at 12-month intervals until study conclusion at 60 months.

There are four specific aims in this study: i) to validate the concept that GastroPanel® biomarker assay is a reliable non-invasive tool in the follow-up of the patients with diagnosed AG; ii) to assess the value of GastroPanel® biomarker profiles as predictors of disease outcome among AG patients during a long-term prospective follow-up; iii) to establish whether any differences in the clinical outcomes exist between HP+ and HP− patients with AG; and finally iv) to validate the novel hypothesis that L-cysteine administered by daily intake of Acetium® capsules (3×200 mg/d) is an effective remedy to induce recovery of the atrophic gastric mucosa.

### Patient Selection

Enrolment of the patients in the study will take place at Hospitals X, Y and Z. Only the patients who have biopsy-confirmed AG of the corpus (AGC) or antrum (AGA) or both (AGP) will be enrolled in the study cohort, classified as mild, moderate or severe, using the USS classification.^{35,36} To be eligible in the cohort, the patient must give a written consent before enrolment in the cohort.

The FlowChart of the patient enrolment in the cohort is illustrated in FIG. 1. In brief, the patients are enrolled in the three clinics among the consecutive patients referred for gastroscopic examination due to varipous indications. Apart from the clinical routine examinations and laboratory tests, all patients are being tested by GastroPanel® for non-invasive diagnosis of their stomach mucosa. All patients testing normal with the GastroPanel® are not eligible for the follow-up cohort, and should be returned to routine management.

### Definition of Atrophic Gastritis

Based on abnormal GastroPanel® test results consistent with AG (AGA, AGC, or AGP), the subjects are allocated to two arms: i) HP+, and ii) HP− patients. Among the HP− patients, only AGC arm is possible, because by definition, AGA diagnosis necessitates the presence of HP. Accordingly, both the AGA and AGC arms only follow the HP+ diagnosis in the FlowChart (FIG. 1).

In both AGA and AGC arms, the biopsy results are correlated with their GastroPanel® biomarker profiles of mild, moderate or severe AG, following the PGI and G-17 cut-off values, respectively, for AGC and AGA. As to AGC, the cut-off for mid AGC is 70 μg/l, for moderate AGC, 50 μg/l, and for severe AGC, 30 μg/l. The likelihood of severe AGA increases with G-17 values below 1.0 pmol/l, moderate and mild AGA typically presenting with G-17 values between 1-2 pmol/l. Importantly, these cut-off values for individual biomarkers are mostly based on studies conducted in low-risk countries for GC and do not necessarily directly applicable to the present setting. More refined data on the cut-off values of individual GastroPanel® biomarkers in different grades of AG will be obtained when the GastroPanel® results are correlated with the biopsy results at study conclusion. This is another important “side result” of this study.

### Age at Study Entry

In principle, there is no specific age limit for the subject to be eligible for this study. Given that AG is a rare condition before age 45-50 years, it is anticipated that the vast majority of the subjects to be enrolled will be above 50 years of age.

### Gender

Both male and female participants will be eligible in the cohort. In this study, every effort is done to minimize any gender selection bias by encouraging both women and men to participate, except those (obviously rare) women who are pregnant, and excluded due to this reason.

### Concomitant Medication, Comorbidity and Exclusion Criteria

Because Acetium® capsules have no known interactions with other drugs, the subjects are generally allowed to continue their regular medication for ailments not related to AG. However, other (eventual) medication targeted to AG should be discontinued prior to the study entry.

Excluded are the following subjects: patients who meet the criteria for medication overuse; patients who have taken anti-psychotics or anti-depressant medications during the previous month; patients who abuse alcohol or other drugs; and potentially fertile and sexually active women who do not practice contraception.

The subjects will be enrolled among the patients who are attending the hospital outpatient departments due to dyspeptic symptoms, which means that the target patients are symptomatic. The intention is to enrol a cohort of subjects with minimum co-morbidity. However, patients with autoimmune-type of disorders (type 1 diabetes, autoimmune thyreoidis, pernicious anemia) are needed to accumulate cases who have AG of autoimmune-type (HP−).

Specific co-morbid medical conditions that exclude participation in this trial include the following categories of patients: severe psychiatric disease, infections, malignancy, short life expectancy, cardiovascular disease, cerebrovascular disease, uncontrolled hypertension, degenerative central nervous system diseases, as well as pregnant and lactating women. In addition, the following patients should be considered non-eligible: 1) the patients whose stomach condition requires surgical treatment, or immediate follow-up treatment for major symptoms, as well as 2) those who refuse to sign the written consent.

### Follow-Up Visits

As shown in the FlowChart (FIG. 1), the subjects enrolled and appropriately allocated to 1) HP+ and 2) HP− arms, with either AGA or AGC, graded as mild, moderate or severe by biopsy, constitute the cohorts to be followed-up prospectively for 5 years (60 months). The enrolled patients are instructed to follow their accustomed dietary practices and use their prescribed medications as usual. All patients are being monitored at the clinics by GastroPanel® assay at 12-month intervals for a total of 5 years, i.e., 6 tests altogether (baseline plus 5 yearly tests), if not randomized for the Acetium intervention arm (see below: 2.4).

### Acetium® Intervention

Whenever GastroPanel® test is indicating the progression of AG (AGC only) during the first 3 years of follow-up, the patient will be randomly allocated to 1) Acetium® intervention arm, or 2) Non-intervention arm (FIG. 1). The criteria for this randomization includes the progression of i) mild AGC to moderate AGC, and ii) moderate AGC to severe AGC, and applies equally to HP+ and HP− study arms. In this trial, we are not interested in testing the subjects with AGA, because the evidence on potential usefulness of Acetium® in these patients is lacking. Accordingly, the randomization applies to AGC patients only.

### Blinding

Because of the fact that no physical placebo is being used, no blinding is needed at the stage of randomization. However, blinded should be the pathologists who examine the biopsies taken on gastroscopy at the end of the study.

### Placebo Control

In this open-label trial, no placebo preparation is used, while those patients allocated to the Non-intervention arm continue their follow-up as do the non-progressors, by repeated GastroPanel testing at 12-month intervals. These patients serve as the study cohort whose AG lesions are being followed-up for the clinical outcomes without medical or other interventions.

### Randomization

Because the subjects eligible for randomization will accumulate over an extended time (any time between the 12-month and 36-month follow-up visits), the subjects will be randomized in relatively small blocks, to avoid the potential bias due to varying the selection criteria over time. In this trial, randomization will be performed using the random number generator (https://www.sealedenvelope.com/simple-randomiser/v1/lists) with block size of 4, and creating unique randomization codes for each study subject.

### Stratification

Randomization alone may not ensure full comparability between participants in the two treatment arms, and stratified randomization is needed to remedy this potential imbalance between the two arms. Of the baseline characteristics of AG that potentially affect the efficacy outcomes in the trials, the most obvious include the following: 1) severity of AG (moderate/severe), 2) localization of AG (antrum/corpus), 3) extent of AG (antrum-only, corpus-only vs. pan-gastritis), and 4) association or not with HP-infection. According to the recommended practice, however, these baseline stratification variables will be entered as covariates in the final multivariate models to control for their potential confounding of the efficacy endpoints.

### Duration of the Acetium® Intervention

In the only published Acetium intervention trial, the patients received Acetium® capsules (100 mg three times a day) for a period of three months.^{62,63} The rational behind this treatment regimen is not clear. L-cysteine administered in this format (Acetium® capsule) is a natural amino acid, with no systemic effects, no known toxicity, and thus no upper limit of daily usage. When used in its original indication (inactivation of acetaldehyde in the stomach after alcohol intake), however, the recommended maximum daily dosage is 1000 mg (10 capsules/day).

Similarly, there are no solid arguments to substantiate the selected 3-month period as the duration of the Acetium® capsule treatment.^{62,63} One could equally well argue that because gastric mucosa has a very short renewal time (under normal conditions), one could anticipate that removing the noxious agent (acetaldehyde) by Acetium® capsules could result in interruption of the Correa cascade and mucosal healing within a much shorter time than 3 months. Equally valid, however, could be the claim that gastric carcinogenesis once initiated by HP and advanced to the stage of AG is a long process, and even after HP elimination and the second trigger (i.e., acetaldehyde) would, indeed, evoke a mucosal recovery that is far less effective than e.g. after early eradication of active HP-infection, and in addition, would necessitate a more prolonged use of Acetium® capsules than just 3 months.

The latter view was adopted in the present trial. To assess the potential efficacy of Acetium® in full-blown AGC, the potential targets of the Acetium® intervention are those with an established progressed AGC. Progression here denotes confirmed progress of mild AGC (at baseline) to the stage of moderate AGC, during the first 3 years of the follow-up. This time limit is set forth to 36 months so as to leave ample of time for the Acetium® intervention, i.e., a minimum of 2 years, given that the total study duration is 5 years. This period of 2+ years sounds reasonably long to give an answer to the question, whether Acetium® intervention (3×200 mg/d; connected with meals), is effective 1) in arresting the progression of AGC, and 2) even induce a recovery of the atrophic mucosa of the stomach corpus.

### Follow-Up Visits and Study Completion

Eligible patients (n=500) are allocated to two major study arms: HP-positive and HP-negative AG. The latter are likely to represent autoimmune-type AG, while the former represent HP-induced AG. The enrolled patients are instructed to follow their accustomed dietary practices and use their prescribed medications as usual, in other words continue spending their normal daily life.

All patients are being monitored by GastroPanel® assay at 12-month intervals for a total of 5 years, i.e., 6 tests altogether (baseline plus 5 yearly tests). The follow-up protocol is the same for the subjects in the Acetium® intervention arm and those in the Non-intervention arm. At study conclusion at 60 months, all patients are subjected to gastroscopy and biopsies. These end-of-study biopsies are read blinded by two expert pathologists, using the updated Sydney System (USS) classification of gastritis. Blinding is particularly important so as NOT to disclose whether the subject belongs in the Non-intervention or Acetium-intervention arm.

### Compliance

Evidence of poor compliance in the prospective cohort studies on patients with chronic HP-infection and/or AG, is not unusual, but long-term cohort studies are still possible to complete successfully.^{14,15,17,18,25,42,43} Therefore, it is crucial to monitor patients' compliance with the follow-up procedures and particularly with the Acetium® intervention issues during the entire 5-year study period. One such approach is the drug or pill count at every follow-up visit, and repeated emphasis on the importance of the adherence to the protocol requirements.

### Study Outcomes

At study conclusion at 60 months, all patients are subjected to the last GastroPanel® test and gastroscopy with biopsies (see biopsy protocol). These end-of-study biopsies are read blinded by two expert pathologists, using the updated Sydney System (USS) classification of gastritis. In the final analysis, these end-of-study biopsies are correlated with the AG status of each patient in baseline biopsy, to define the 3 potential (mutually competing) outcomes: 1) No progression; 2) Progression (increase of at least one AG grade or AG extension to another topographic area), and 3) Regression (decrease of at least one AG grade or AG disappearance of another topographic area). This applies basically to both the Non-intervention and Acetium®-intervention arms. In the latter arm, the outcome 1) can equally well be defined as disease arrest, if AG progression is being halted by Acetium® intervention. If the intervention is associated with regressive outcome 3), one can speak about mucosal recovery or healing. Similar as in the Non-intervention arm, all three outcomes can be separately assessed in HP+ and HP− arms, giving an insight on eventual differences in AG outcomes between these two disease categories.

### Methods

The general outline of the study and the trial design have been detailed above. The following methods described here are used to examine the patients and their samples at the baseline visit and follow-up visits, following the study protocols.

### Gastroscopy and Biopsy Procedures for Eligibility of the Patients

According to the Flowchart (FIG. 1), gastroscopy and biopsies is the first-line diagnostic procedure for these gastroscopy-referral patients. Only the patients disclosing AG in the biopsies are potentially eligible for the study, and will be interviewed for possible exclusion criteria before enrolled in the study. At baseline, also GastroPanel® test is made to control the gastric biomarker status, which consists an important baseline measurement to be used as the reference in the Acetium® intervention arm (see later).

As per FlowChart (FIG. 1), the follow-up of the study subjects in both HP+ and HP− arm as well as in the Non-intervention and Acetium-intervention groups, is based exclusively on GastroPanel® testing, repeated at 12-month intervals. Only at the last follow-up visit concluding the study, all subjects will undergo a routine gastroscopic examination, which will be complemented by biopsy sampling from the antrum and corpus, according to the principles of the USS.

### Biopsy Protocols

On endoscopy (esophago-gastro-duodenoscopy), all observed abnormal mucosal lesions are noted and photographed, and if necessary (e.g. suspicion of malignancy) subjected to additional biopsies. The optimal biopsy protocol follows the USS classification. In each patient, routine biopsy specimens are taken from the antrum and corpus, at least two biopsies from each. These biopsies are taken from the large and small curvature of the middle antrum and from the large curvature of the corpus. In addition, two extra biopsies are recommended to be taken from the incisura angularis. Importantly, to facilitate the pathology reading, the biopsies from the antrum and incisura (biopsies 1, 2, 3 and 4) must be immersed into one and the same formalin tube, and embedded into the same paraffin block (Block No. 1; labeled ANTRUM). The two biopsies from the corpus are put into another formalin tube, and embedded into the same paraffin block (Block No. 2; labeled CORPUS).

### Preparation of Microscopy Slides

The biopsies from formalin bottles/tubes are embedded in paraffin using the routine procedures at the Pathology Laboratory of the Hospital. The blocks are cut into 4-μ-sections, and stained with hematoxylin eosin (HE) for routine diagnosis and with modified Giemsa for identification of HP in the specimens.

### Interpretation of the Biopsies

All gastroscopy biopsies are examined by the expert pathologists at Hospital X, among the daily routine samples. The diagnoses are reported using the USS for classification of gastritis, and diagnosed into different “phenotypes” of gastritis.

The topography of the gastritis is considered fundamental in this classification. Accordingly, the gastritis restricted to the antrum, restricted to the corpus, or a pangastritis should be reported separately. The etiology of gastritis, if known, is to be added to the diagnosis as a prefix (e.g. HP antrum gastritis; autoimmune corpus gastritis, etc.). As a suffix, one should give a grading for each five morphological variables shown in ANNEX 6. These are; 1) chronic inflammation (chronic gastritis); 2) the activity of the gastritis measured by the presence of polymorphonuclear leucocytes in the inflammatory infiltrate; 3) intestinal metaplasia (IM); 4) atrophy manifested by the loss of the normal mucosal glands; and 5) the presence of HP organisms. The guidelines recommend these five parameters to be recorded separately for both antrum and corpus, and semi-quantitatively graded as absent, mild, moderate or severe.

### GastroPanel® Screening for the Baseline Biomarker Status

GastroPanel® test is the first-line diagnostic test used for the initial screening of the potential study subjects, concomitantly with their gastroscopic examination. Due to its extremely high negative predictive value (NPV),⁵²⁻⁵⁶ a normal result in GastroPanel® test excludes AG whereas test results suggesting AG (antrum, corpus or both) have a close correlation with the biopsies. During the follow-up visits at 12-month intervals, only the non-invasive GastroPanel® is used to monitor the gastric mucosal status, with particular reference to the clinical outcomes of AG (FIG. 1).

GastroPanel® is a user-friendly ELISA technique, consisting of a panel of four biomarkers specific for the gastric mucosa: 1) Pepsinogen I (P-PGI), 2) Pepsinogen II (P-PGII), 3) Gastrin-17 (P-G-17) and 4) HP-antibody (P-HpAb). The current test version is called Unified GastroPanel, where all its biomarkers are processed using unified processing conditions in the ELISA automation.

### ELISA Test for Pepsinogen I and Pepsinogen II

P-PGI is secreted solely by the chief cells (chief cell/mucous neck cells) of the corpus mucosa. Atrophic corpus gastritis leads to a loss of these cells and, as a result, the P-PGI level in circulation decreases. P-PGII is produced by the chief cells and mucous neck cells of the gastric mucosa, by pyloric glands in the gastric antrum and by Brunner's glands in the proximal duodenum. The ratio of PGI to PGII concentration in the plasma of normal subjects is above 3.0. While not secreted by any other cells at any other anatomic sites, these two biomarkers are specific for gastric mucosa, i.e., stomach-specific biomarkers.

In the GastroPanel® test, P-PGI and P-PGII biomarkers are determined according to the instructions of the manufacturer from a plasma samples. Pepsinogen I ELISA kit (Biohit Cat. No. 601 010.01), Pepsinogen II ELISA kit (Biohit Cat. No. 601 020.01). Both P-PGI and P-PGII ELISA is based on a sandwich enzyme immunoassay technique with PGI-and PGII-specific capture antibody, adsorbed on a microplate, and the detection antibody labeled with horseradish peroxidase (HRP).

### ELISA Test for Gastrin-17

G-17 is secreted exclusively by the gastrin-cells (G-cells) in the antrum, representing a fraction of the total gastrin concentration in the circulation. When dormant, the G-cells secrete only small amounts of G-17 hormone. The maximal secretion is achieved after physiological protein stimulation, or when the acid secretion in the stomach is low or absent. As a result of antral atrophy (i.e., loss of glands), the amount of G-cells decreases and, consequently, both the basal and post-prandial secretion of gastrin decreases. The G-17 ELISA method in the GastroPanel® is specific to “amidated” G-17 molecule, which is the most important member of the gastrin/cholecystokinin-family, regulating the physiology of the upper gastrointestinal tract.

### Stimulation of Gastrin-17

If the GastroSoft® report from the fasting sample implicates AG in the antrum, it is recommended to repeat Gastrin-17 test in a post-prandial blood sample. The secretion of G-17 can be stimulated by the intake of a protein drink having average protein content of 77% [Biohit Cat. No. 601038 (50×20 g), Cat. No. 601037 (5×20 g)]. This stimulation should not be performed for patients who are sensitive to lactose (i.e., lactose intolerance or hypolactasia). To prepare the protein juice, 20 g of protein (one foil bag of protein powder) is mixed with 150 ml of water. The stimulated (postprandial) blood sample must be taken 20 minutes after the intake of the protein juice.

### ELISA Test for Helicobacter pylori

GastroPanel® test for H. pylori is performed from the plasma samples. The test is based on an enzyme immunoassay technique, with purified HP bacterial antigen, adsorbed on a microplate, and a detection antibody labeled with horseradish peroxidase (HRP).

### Patient Preparation for GastroPanel® Test

Reliable results from the GastroPanel® examination necessitate some preparatory measures of the patient. Detailed instructions are given to each test subject at the time of his/her consenting to participate in the GastroPanel screening phase (FIG. 1).

Proper conduction of and reliable results from the GastroPanel® examination necessitate some preparatory measures of the patient. Detailed instructions are usually given to each test subject at the time of his/her consenting to participate, but this does not apply here, because all subjects already complete the preparation for gastroscopy. Their compliance with the taking of medicines listed below will be controlled before taking the blood sample.

The patient should not drink, eat or smoke for at least 4 hours before the sample collection, e.g., 10-hour fasting overnight is perfect. The patients are allowed to take their prescribed, regular medication. However, it is necessary to report any use of proton pump inhibitors (PPIs, such as Esomeprazole, Lanzoprazole, Omeprazole and Rabeprazole), and the time of discontinuation in PPI use) on the Request Form (ANNEX 2), because these medicines interfere with the output of GastroPanel® biomarkers (www.gastropanel.com/ GastroPanel® Sample collection Instructions).

### Sample Collection for GastroPanel® Test

For each patient, 2 plasma tubes will be taken for GastroPanel® test. Additionally, Gastrin-17 can be determined also after stimulation (see 3.1.3.). A minimum of 2 ml EDTA plasma from a fasting blood sample is taken into an EDTA tube (e.g. Biohit Cat. no. 454235 Vacuette 4 ml tube containing K2EDTA). Use of Gastrin-17 stabilizer 100 μl/2 ml plasma (Biohit Cat. No. 601 050 or 601 051) allows the sample transfer at room temperature (20-25° C.), and permits the ELISA tests within 4 days from the sample collection.

### Sample Processing

The blood sample needs to be centrifuged within 30 minutes, at 1800-2000 g for 10 minutes (e.g. Vacuette, Biohit Cat. no. 454235) or as prescribed by tube manufacturer or centrifuge manufacturer (e.g. StatsSpin Express 2, at 4000 g for 2 minutes). Unless immediately used for testing, the EDTA plasma needs to be frozen instantly (−70° C.). Preferable storage temperature of the sample with the Gastrin-17 stabilizer is in the refrigerator at 2-8° C., for up to 4 days. If the sample cannot be analysed within 4 days, it should be stored frozen at −15 to −20° C., but for any storage of over 2 weeks, the temperature should be −70° C.

The samples should be mixed thoroughly after thawing. Multiple freezing and thawing cycles should be avoided. Lipemic or cloudy specimens must not be used. If a postprandial blood sample is needed, it should be taken into an EDTA tube after 20 minutes upon the intake of the protein drink. For further details, refer to the section describing Gastrin-17 stimulation (see www.biohithealthcare.com/GastroPanel Sample Collection Instructions and below).

### Evaluation of GastroPanel® Results by GastroSoft® Application

Prerequisite for reliable results is an adequate EDTA plasma sample, taken following the manufacturer's instructions for sampling (above) and for conducting the ELISA tests. The results of the GastroPanel® examination are evaluated using the GastroSoft® software. The principles and algorithm used by the GastroSoft® software is based on the Updated Sydney System (USS) for classification of gastritis. The comprehensive biomarker profiles and the diagnostic equivalents in the USS are summarized in Table 1, which also illustrates the other clinical conditions (gastric functional abnormalities) diagnosed by the specific biomarker profiles.

TABLE 1
GastroPanel ® biomarker profiles define gastritis phenotype*

| | GastroPanel ® Biomarkers | | | | | | |
|---|---|---|---|---|---|---|---|
| | | | | | | H. pylori | |
| | | | | | | IgG | |
| | | | PGI/PGII | | | Antibody | |
| Marker | Pepsinogen I | Pepsinogen II | Ratio | Gastrin-17b | Gastrin-17s | titer | |
| Profile | (30-160 μg/l)@ | (3-15 μg/l) | (3-20) | (1-7 pmol/l) | (3-30 pmol/l) | (<30 EIU) | Interpretation |
| 1 | N | N | N | N | N | N | Healthy mucosa |
| | | | | | | | (no atrophy, no |
| | | | | | | | Hp- infection) |
| 2 | N | N | N | L* | N | N | Healthy mucosa. |
| | | | | | | | High acid output. |
| 3 | N or H\hat{ } | N or H\hat{ } | N | H** | N | N | Healthy mucosa. |
| | | | | | | | Low acid output |
| | | | | | | | due to e.g. PPI |
| | | | | | | | medication |
| 4a | N or H\hat{ } | N or H\hat{ } | N | N or H\hat{ } | ND | H | Active Hp |
| | | | | | | | infection, not |
| | | | | | | | treated |
| 4b | N | N | N | N | ND | N or H† | Hp infection |
| | | | | | | | successfully |
| | | | | | | | eradicated |
| 4c | N | H | N | H | ND | H | Hp eradication |
| | | | | | | | failed |
| 5 | L | L | L | H | ND | N\hat{ }\hat{ } or H | Atrophic gastritis |
| | | | | | | | in corpus and |
| | | | | | | | fundus (AGC) |
| 6 | N | N | N | L | L | H | Atrophic gastritis |
| | | | | | | | in antrum (AGA) |
| 7 | L | L | L | L | L | N\hat{ }\hat{ } or H | Atrophic gastritis |
| | | | | | | | in both antrum |
| | | | | | | | and corpus (AGP) |
| 8 | H | H | N | H | ND | N | Short (4-10 d) |
| | | | | | | | pause in |
| | | | | | | | continuous PPI |
| | | | | | | | treatment. |
| | | | | | | | Rebound in |
| | | | | | | | gastric acid |
| | | | | | | | output. |

*Classification of gastritis (AGA, AGC, AGP) by GastroSoft ® is based on Updated Sydney (USS) Classification.
N = normal;
L = low; H = high;
*Test PPI medication for two weeks, G17b should normalize;
**Stop PPI medication,
G-17b should normalize within two weeks;
ND, no need for testing;
\hat{ }PGI, PGII and G-17 can be elevated due to mucosal inflammation;
\hat{ }\hat{ }Hp antibodies can disappear in mucosal atrophy with protracted clinical course;
@Pepsinogen I cut-off value 30 μg/l is consonant with moderate/severe AG;
†Hp antibody levels can remain elevated for months after successful eradication of Hp.

### Statistical Analyses

All statistical analyses will be performed using the SPSS 27.0.1.0 for Windows (IBM, NY, USA) and STATA/SE 17.0 software (STATA Corp., Texas, USA). The descriptive statistics will be conducted according to routine procedures. Frequency tables will be analysed using the χ2-test, with the likelihood ratio (LR) or Fisher's exact test for categorical variables. Differences in the means of continuous variables (e.g. biomarker levels) are analysed using non-parametric (Mann-Whitney or Kruskal-Wallis) test for two-and multiple independent samples, respectively.

### Follow-Up in the Non-Intervention Group

In the basic study setting, this longitudinal cohort study is designed to examine the clinical outcomes of AG without intervention during a prospective follow-up of 5 years. These clinical outcomes are only disclosed at study conclusion, when gastroscopy and biopsies will be available. In the final analysis, these end-of-study biopsies are compared with the baseline serological AG status of each patient that enables classification into one of 3 potential (mutually competing) outcomes: 1) No progression; 2) Progression, and 3) Regression.

### Life-Table Techniques

The longitudinal study design with clearly established clinical outcomes (study endpoints) makes the data amenable to analysis by different life-table techniques can be used to compare the two study arms (HP+and Hp− cases). In the most simple form, this is done using univariate survival (Kaplan-Meier) analysis, where i) either the AG progression event (at any follow-up visit) or ii) AG regression event detected by the respective changes in the GastroPanel biomarker profiles, is used as the endpoint in K-M analysis. The follow-up dates represent the event of interest, and the stratum-specific (HP+ vs. HP−) estimates are calculated using the log-rank (Mantel-Cox) statistics. The same approach can be used to calculate the difference in the duration of attack abortion (i.e., time to progression or regression) between the two study arms. The independent effect of Acetium can be taken into account already here, using the multivariate Cox proportional hazards regression models, where all recorded baseline characteristics of the patient (Questionnaire) can be entered as covariates.

### Generalised Linear Models (GEE and Panel Poison)

In addition, using the AG progression or AG regression as the event of interest, the effects of HP+ and HP− can be modelled also using the regression techniques based on count variables, i.e., Poisson regression. In this approach, AG regression or AG progression are expressed as events per person time (months) at risk, and the two arms (HP+ and HP−) can be compared using the incidence rate ratio (IRR) statistics. When applied to panel type of data recorded at each FU visit (panel Poisson), all covariates (biomarker levels) subject to random intra-subject variation (by FU visits) can be adequately controlled in this longitudinal setting. A similar type of approach based on panel data, i.e., generalized estimating equation (GEE) modelling, can be used to estimate the effect of HP (HP+/HP−) on persistence (=no regression, no progression) of AG, using the (persistence dichotomized yes/no) recorded at each FU visit as the dependent variable, and adjusted for potential confounders in multivariate GEE. A wide variety of such potential confounders should be examined, as recorded in the baseline questionnaire.

### Modelling of the AG Outcomes by Competing-Risks Regression

In all study settings where repeated measures involve the same subjects, the results tend to be correlated. In other words, the probability of recording a persistent AG is greater among the subjects who reported a persistent AG (no change to baseline) in the previous follow-up visit, if the record is repeated within a reasonable time (in this case on annual basis). Statistical techniques that i) fail to take these correlations into account are not valid, and ii) methods that do not exploit all the recorded data (in a repeated measures setting) would be inefficient. Marginal (GEE, Panel Poisson)-and mixed-effects models are both capable of handling these issues, showing a greater efficiency as compared with standard logistic regression and Cox models for studying the clinical outcomes of AG. Both methods are technically suitable for analysis of the panel data of the present study setting, but because they only accept a binomial (yes/no) dependent variable, this would necessitate a separate analysis for each of the multiple study outcomes (No progression, Progression, Regression).

Natural history study for AG is more complex than merely showing a progression (Correa Cascade) vs. no progression as dichotomous (yes/no) outcome. If carefully modeled, it can be anticipated that there are three 1 possible outcomes to be observed during the AG follow-up study, which should be treated as competing (mutually exclusive) events. These include the following: i) no change in AG (=AG grade remain unchanged as compared with the baseline), ii) progression of AG (=increase in AG grade from the baseline status), iii) regression (=AG grade less severe at study conclusion than at baseline).

If the i) the longitudinal data be utilized in full, ii) intra-subject variation of the repeated measurements at FU visits be taken into account, and iii) the multiple-outcome dependent variable (no change, progression, regression) be treated in a single statistical model, we need to apply a special technique, known as competing-risks regression,^{78,79} This elegant technique can be ALSO used to model the impact of Acetium® intervention on the competing risks outcomes of this trial, adjusted for other covariates as potential confounders. The major advantage to conventional (Cox) models is that while usual censoring (exclusion) from the study in Cox prevents you from observing the event of interest, a competing event prevents the occurrence of the event of interest. In simple terms, competing-risks regression generates hazard for the events of interest, while simultaneously keeping the subjects who experience competing events still “at risk” so that they can be adequately counted and have no chance of failing.^{78,79}

### Acetium-Intervention

Nested within a longitudinal cohort study, the Acetium® intervention can be considered a randomized controlled trial (RCT) testing the efficacy of Acetium® capsules (slow-release L-cysteine) in restoring the structure and function of gastric mucosa among patents with HP-associated (HP+)- or autoimmune type (HP−) AG, shown to make progression during the follow-up. The null hypothesis of the study implicates that the intake of Acetium® capsules is no better than no intervention in restoring the physiological functions of the stomach mucosa, and recovery of AG (arrest the disease, or mucosal healing).

### Study Endpoints

Rejection or not of the null hypothesis is based on comparison of the two strata (Acetium® and no intervention) against two primary study endpoints (efficacy measures): 1) Changes in the serum levels of the relevant stomach-specific biomarkers (GastroPanel test) from the baseline values (diagnostic to AG), towards (or falling within) their reference (normal) values; and 2) Biopsy-confirmed recovery of atrophic gastric mucosa by at least one histological grade (e.g. from severe to moderate AG; moderate to mild AG), based on a blinded reading of the biopsies taken at study conclusion, as compared to each subject's mucosal status at baseline (GastroPanel® test).

### Conventional Statistical Techniques

The descriptive statistics will be conducted according to routine procedures. Frequency tables will be analyzed using the χ2-test, with the likelihood ratio (LR) or Fisher's exact test for categorical variables. Standard statistics are used to compare the efficacy of the two study arms on the observed changes in the serum levels of the GastroPanel® biomarkers before and after Acetium® intervention. The effects of test preparation and non-intervention can be analyzed separately in non-parametric paired samples t-test (Wilcoxon signed ranks test) by comparing the pairs of the baseline- and post-trial values. Another approach is to calculate the effect size in both arms (i.e., increase/decrease of the biomarker levels by Acetium treatment as compared with the baseline) and to compare this effect size to that observed in the non-intervention arm.

### Univariate and Multivariate Logistic Regression Models

To compare categorical binary outcomes (arrest/recovery; Y/N) between Acetium-intervention and non-intervention arm, conventional regression models can be used, where the results are expressed as crude OR (odds ratio), and their 95% confidence intervals (95% CI). The independent effect of Acetium® (adjusted for potential confounders) can be analysed using the multivariate logistic regression model, where all recorded baseline characteristics (gender, age, etc) can be entered as covariates, including the 4 stratification variables: i) AG severity, ii) AG localization, iii) extent of AG, iv) HP-association. For categorical binary endpoints (arrest/recovery; Y/N), the results of these multivariate models are expressed as adjusted OR, and their 95% CI.

### Test Performance Indicators and Longitudinal Predictive Value of the Biomarkers

Of secondary importance in this study are the performance indicators (sensitivity, specificity, positive predictive value, PPV, negative predictive value, NPV and their 95% CI) of individual biomarkers (PGI, PGI/PGII, G-17) and GastroPanel® test diagnostic report to predict the appropriate endpoints (AGA, AGC) at study conclusion. These are calculated using the STATA/SE software and the diagti algorithm introduced by Seed et al. (2001). This algorithm also calculates the area under ROC (Receiver Operating Characteristics) called AUC, for each biomarker. Because GastroPanel® is a quantitative ELISA test, these ROC curves can be used to identify the optimal sensitivity/specificity balance that gives each biomarker an optimal detection of the study endpoint (AG grades, localization, extent). Significance of the difference between AUC values can be estimated using STATA's roccomb test with 95% CI.

Of interest is the value of individual GastroPanel® biomarkers as predictors of AG progression during a long-term follow-up, Prompted by two recent studies, where GastroPanel® biomarkers were shown to be powerful long-term predictors of GC.^{74,75} The current study design offers perfect possibilities to assess, whether the below-cut-off values of G-17, PGI, and PGI/PGII are of predictive value for progression of AGA and AGC, respectively. Following the definition of the 3 clinical outcomes (no change, regression, progression), the calculation is done by conventional univariate regression (using non-progressors as reference) and the result expressed as OR, with 95% CI. If more sophisticated approach is indicated, i.e., any marker showing significant predictive value, a nested case-control setting can be designed with 1:1 ratio of cases (progressors) and controls selected among the non-progressors, matched with age and gender, and analysed using conditional logistic regression to control for the potential confounders.⁷⁴

### Power Analysis

Due to the complexity of the study design, several options exist to estimate the statistical power of the study. These are described shortly in the following.

### Follow-Up of the Non-Intervention Group for the Clinical Outcomes of AG

In the statistical analysis of this part of the longitudinal study, both conventional techniques (e.g. Kaplan-Meier (log-rank) test, Cox proportional hazards) and more sophisticated methods (competing risks regression) will be used.

In this setting, the power of the study is most simply calculated based on the estimates of the progression rates of e.g. mild AGC during the follow-up, assessed by life-table (Kaplan-Meier) analysis. Using the Kaplan-Meier (log-rank, Mantel-Cox) comparison statistics for the two study arms (HP− and HP+), this study would be adequately powered (Type II error 0.80, type I error 0.05) to detect a true difference of 10% in the progression rates, if there are 252 patients in the HP+ and 252 in HP− arms. With such a cohort of 504 subjects (with equal numbers in HP+ and HP− arms), this study is adequately powered for a true difference in the AGC progression rates of 20% (rate 0.2) in one arm and 30% (rate 0.3) in the other arm. An uneven distribution of the study subjects in HP+ and HP− arms, will erode the study power, however, while any increase in effect size will increase the power as well.

### Acetium-Intervention

Because of the fact that GastroPanel® is composed of 4 stomach-specific biomarkers, of which 3 are relevant for this purpose (PGI, PGII, G-17), and the combination of which is the key for appropriate diagnosis of the endpoints (AGA, AGC), calculating the study power is not straightforward. In principle, it should be calculated separately for the biomarkers of AGC (PGI, PGII and PGI/PGII ratio) and those that implicate AGA (G-17).

Given that there are no figures available for biomarker level fluctuations following any intervention, we must base the power calculations for the changes in biomarker values (after Acetium® treatment) that are reported in the preliminary study from Italy.^{62,63} Importantly, however, this study only included cases with AGC (with low PGI & PGI/II ratio and elevated G-17), but not patients with AGA (where G-17 is low and PGI within normal range). Thus, we calculated the power of this study on the basis of these corpus-specific biomarkers only, and used these to estimate the appropriate cohort size needed in both the Acetium® and non-intervention arms.

Accordingly, the Italian study reported a post-treatment (3-month) increase of PGI levels from 7.9 μg/L to 11.4 μg/L (difference of 3.5 μg/L). Unfortunately, the SDs for the two values were not reported, but based on estimates from another cohort (n=42) of AG (corpus) patients, with similar baseline PGI values, these SD values are estimated as 7.5 and 10 μg/L, respectively. Using the 2-sample mean test for paired samples, this study would be adequately powered (Type II error 0.80, type I error 0.05) to detect a true difference in PGI increase of this magnitude (3.5 μg/L), if there are 55 patients in the Acetium® study arm. This estimate is sensitive to SDs as well as correlation between the paired samples. If the SD increases, a larger cohort size is needed, but if the correlation is higher than the default 0.5, a markedly smaller cohort size is enough.

To be on the safe side, a cohort of 60 patients would give the power of 84% to detect this difference in PGI increase after Acetium® treatment, and because of this, the aim of the current randomizations is to reach a cohort of 60 subjects in both the Acetium® and non-intervention arm (ANNEX 1). With the cohort of 120 subjects, randomized 1:1 into Acetium® and non-intervention arms, and using two-sample mean test (for independent samples) for the effect differences (i.e., PGI increase during intervention), we enter up with the estimates that this study is adequately powered (Type II error 0.80, type I error 0.05) to detect a true difference in effect size estimates (pre-trial to post-trial) as follows: 3.5 μg/L (Acetium® arm) and 2.15 μg/L (non-intervention arm), with assumed SD of 3.0 and 2.0, respectively. Even a slightly smaller effect in the non-intervention arm would increase the study power close to 100%, allowing also a much wider range of SD values.

### CITATION LIST

[1] Talley N J, Zinsmeister A R, Schleck C D, Melton III L J. Dyspepsia and dyspepsia subgroups: a population-based study. Gastroenterology 1992;102:1259-1268.
[2] Drossman D A, Li Z, Andruzzi E, Temple R D, Talley N J, Thompson W G, et al. U.S. householder survey of functional gastrointestinal disorders. Prevalence, socio-demography and health impact. Dig Dis Sci 1993;38:1569-1580.
[3] Hansen J M, Bytzer P, Schaffalitzky De Muckadell O B. Management of dyspeptic patients in primary care. Value of the unaided clinical diagnosis and of dyspepsia subgrouping. Scand J Gastroenterol 1998;33:799-805.
[4] Heikkinen M, Pikkarainen P, Takala J, Julkunen R. General practitioners' approach to dyspepsia. Survey of consultation frequencies, treatment, and investigations. Scand J Gastroenterol 1996;31:648-653.
[5] Majumdar S R, Soumerai S B, Farraye F A, Lee M, Kemp J A, Henning J M, et al. Chronic acid-related disorders are common and under investigated. Am J Gastroenterol 2003;98:2409-2414.
[6] Nebel O T, Fornes M F, Castell D O. Symptomatic gastroesophageal reflux: incidence and precipitating factors. Am J Digest Dis 1976;21:953-956.
[7] Malfertheiner P, Megraud F, O'Morain C A, Atherton J, Axon A T, Bazzoli F, Gensini G F, Gisbert J P, Graham D Y, Rokkas T, El-Omar E M, Kuipers E J. European Helicobacter Study Group. Management of Helicobacter pylori infection-the Maastricht IV/Florence Consensus Report. Gut 2012;61:646-664.
[8] Sipponen P, Kekki M, Haapakoski J, Ihamäki T, Siurala M. Gastric cancer risk in chronic atrophic gastritis: statistical calculations of cross-sectional data. Int J Cancer 1985;35:173-177.
[9] Israel D A, Peek R M. Review article: pathogenesis of Helicobacter pylori-induced gastric inflammation. Aliment Pharmacol Ther 2001;15:1271-1290.
[10] Parsonnet J, Hansen S, Rodriguez L, Gelb A B, Warnke R A, Jellum E. Helicobacter pylori infection and gastric lymphoma. N Engl J Med 1994;330:1267-1271.
[11] Khulusi S, Mendall M A, Patel P, Levy J, Badve S, Northfield T C. Helicobacter pylori infection density and gastric inflammation in duodenal ulcer and non-ulcer subjects. Gut 1995;37:319-324.
[12] International Agency for Research on Cancer, World Health Organization. Schistosomiasis, liver flukes and Helicobacter pylori. IARC working group on the evaluation of carcinogenic risks to human. Monogr Eval Carcinog Risks Hum 1994;61:218-220.
[13] Uemura N, Okamoto S, Yamamoto S, Matsumura N, Yamaguchi S, Yamakido M, et al. Helicobacter pylori infection and the development of gastric cancer. N Engl J Med 2001;345:829-832.
[14] Valle J, Kekki M, Sipponen P, Ihamäki T, Siurala M. Longterm course and consequences of Helicobacter pylori gastritis. Results of a 32-year follow-up study. Scand J Gastroenterol 1996;31:546-550.
[15] Kuipers E J, Uyterlinde A M, Peña A S, Roosendaal R, Pals G, Nelis G F, et al. Long-term sequelae of Helicobacter pylori gastritis. Lancet 1995;345:1525-1528.
[16] Correa P, Haenszel W, Cuello C, Zavala D, Fontham E, Zarama G, et al. Gastric precancerous process in a high risk population: cohort follow-up. Cancer Res 1990;50:4737-4740.
[17] Filipe M I, Munoz N, Matko I, Kato I, Pompe-Kirn V, Jutersek A, et al. Intestinal metaplasia types and the risk of gastric cancer: a cohort study in Slovenia. Int J Cancer 1994;57:324-329.
[18] Ohata H, Kitauchi S, Yoshimura N, Mugitani K, Iwane M, Nakamura H, et al. Progression of chronic atrophic gastritis associated with Helicobacter pylori infection increases risk of gastric cancer. Int J Cancer 2004;109:138-43.
[19] Correa P. A human model of gastric carcinogenesis. Cancer Res 1988;48:3554-3560.
[20] Correa P. The epidemiology of gastric cancer. World J Surg 1991; 15:228-234.
[21] Correa P. Human gastric carcinogenesis: a multistep and multifactorial process. First American Cancer Society Award Lecture on Cancer Epidemiology and Prevention. Cancer Res 1992;52:6735-6740.
[22] Weck M N, Stegmaier C, Rothenbacher D, Brenner H. Epidemiology of chronic atrophic gastritis: population-based study among 9444 older adults from Germany. Aliment Pharmacol Ther 2007;26:879-887.
[23] Lomba-Viana R, Dinis-Ribeiro M, Fonseca F, Vieira A S, Bento M J, Lomba-Viana H. Serum pepsinogen test for early detection of gastric cancer in a European country. Eur J Gastroenterol Hepatol 2012;24:37-41.
[24] Bornschein J, Selgrad M, Wex T, Kuester D, Malfertheiner P. Serological assessment of gastric mucosal atrophy in gastric cancer. BMC Gastroenterol 201212:10. doi: 10.1186/1471-230X-12-10.
[25] Dinis-Ribeiro M, Areia M, de Vries A C, Marcos-Pinto R, Monteiro-Soares M, O'Connor A, Pereira C, Pimentel-Nunes P, Correia R, A. Ensari A, Dumonceau M, Machado J C, Macedo G, Malfertheiner P, Matysiak-Budnik T, Megraud F, Miki K, O'Morain C, Peek R M. Ponchon T, Ristimaki A, Rembacken B, Carneiro F, Kuipers E J, and behalf of MAPS Participants. Management of precancerous conditions and lesions in the stomach (MAPS): guideline from the European Society of Gastrointestinal Endoscopy (ESGE), European Helicobacter Study Group (EHSG), European Society of Pathology (ESP), and the Sociedade Portuguesa de Endoscopia Digestiva (SPED). Endoscopy 2012; 44:74-94.
[26] Ihamäki T, Saukkonen M, Siurala M. Long term observation of subjects with normal mucosa and with superficial gastritis: results of 23-27 years follow-up examination. Scand J Gastroenterol. 1978; 13:771-775.
[27] Ormiston M C, Gear M W, Codling B W. Five year follow-up study of gastritis. J Clin Pathol. 1982;35:757-760.
[28] Kapadia C R. Gastric atrophy, metaplasia and dysplasia. A clinical perspective. J Clin Gastroenterol. 2003; 36:29-36.
[29] Laurén P. The two histological main types of gastric carcinoma: diffuse and so-called intestinal type carcinoma. Acta Pathol Microbiol Scand. 1965; 64:31-49.
[30] Fukao A, Hisamichi S, Ohsato M, et al. Correlation between the prevalence of gastritis and gastric cancer in Japan. Cancer Causes Control. 1993; 4:17-20.
[31] Genta R M. Review article: gastric atrophy and atrophic gastritis-nebulous concepts in search of a definition. Aliment Pharmacol Ther. 1998; 12:17-23.
[32] Dixon M F, Genta R M, Yardley J H, et al. Classification and grading of gastritis. The updated Sydney System. International Workshop on the Histopathology of Gastritis, Houston 1994. Am J Surg Pathol. 1994; 20:1161-1181.
[33] Misiewicz J J. The Sydney System: a new classification of gastritis. Introduction. J Gastroenterol Hepatol. 1991; 6:207-208.
[34] Plummer M, Buiatti E, Lopez G, et al. Histological diagnosis of precancerous lesions of the stomach: a reliability study. Int J Epidemiol. 1997; 26:716-720.
[35] Dixon M F, Genta R M, Yardley J H, Correa P. Classification and grading of gastritis. The updated Sydney System. International Workshop on the Histopathology of Gastritis, Houston 1994. Am J Surg Pathol. 1996;20:1161-1181.
[36] Sipponen P, Price A B. The Sydney System for classification of gastritis 20 years ago. J Gastroenterol Hepatol. 2011;26: Suppl 1:31-34.
[37] Marcos-Pinto R, Carneiro F, Dinis-Ribeiro M, Wen X, Lopes C, Figueiredo C, Machado J C, Ferreira R M, Reis C A, Ferreira J, Pedroto I, Areias J. First-degree relatives of patients with early-onset gastric carcinoma show even at young ages a high prevalence of advanced OLGA/OLGIM stages and dysplasia. Aliment Pharmacol Ther. 2012;35:1451-1459.
[38] Correa P. Clinical implications of recent developments in gastric cancer pathology and epidemiology. Semin Oncol. 1985;12:2-10.
[39] Genta R M, Rugge M. Gastric precancerous lesions: heading for an international consensus. Gut. 1999; 45:15-18.
[40] Guindi M, Riddell R H. The pathology of epithelial pre-malignancy of the gastrointestinal tract. Best Pract Res Clin Gastroenterol. 2001;15:191-210.
[41] Riddell R H. Premalignant and early malignant lesions in the gastrointestinal tract: definitions, terminology and problems. Am J Gastroenterol. 1996;91:864-872.
[42] Rugge M, Farinati F, Di Mario F, et al. Gastric epithelial dysplasia: a prospective multicenter follow-up study from the Interdisciplinary Group on Gastric Epithelial Dysplasia. Hum Pathol. 1991;22:1002-1008.
[43] Rugge M, Baffa R, Farinati F, et al. Epithelial dysplasia in atrophic gastritis. Bioptical follow-up study. Ital J Gastroenterol. 1991;23:70-73.
[44] Riddell R H, Goldman H, Ransohoff D F, et al. Dysplasia in inflammatory bowel disease: standardized classification with provisional clinical applications. Hum Pathol. 1983;14:931-968.
[45] Lauwers G Y, Shimizu M, Correa P, et al. Evaluation of gastric biopsies for neoplasia: differences between Japanese and Western pathologists. Am J Surg Pathol. 1999;23:511-518.
[46] Lauwers G Y, Carneiro F, Graham D Y., et al. Gastric carcinoma. In: Bosman F T, Carneiro F, Hruban R H, Theise N D. (Eds). WHO Classification of tumours of the digestive system. 4^{th} Ed. IARC Press; Lyon: 2010. p. 48-58.
[47] Gutiérrez-González L, Wright N A. Biology of intestinal metaplasia in 2008: more than a simple phenotypic alteration. Dig Liver Dis. 2008;40:510-522.
[48] Eriksson N K, Färkkilä M A, Voutilainen M E, et al. The clinical value of taking routine biopsies from the incisura angularis during gastroscopy. Endoscopy 2005;37:532-536.
[49] Guarner J, Herrera-Goepfert R, Mohar A, et al. Diagnostic yield of gastric biopsy specimens when screening for preneoplastic lesions. Hum Pathol. 2003;34:28-31.
[49b] den Hollander W J, Holster I L, den Hoed C M, Capelle L G, Tang T J, Anten M P, Prytz-Berset I, Witteman E M, Ter Borg F, Hartog G D, Bruno M J, Peppelenbosch M P, Lesterhuis W, Doukas M, Kuipers E J, Spaander M C W. Surveillance of premalignant gastric lesions: a multicentre prospective cohort study from low incidence regions. Gut 2019;68:585-593.
[50] Miki K, Ichinose M, Shimizu A, Huang S C, Oka H, Furihata C, Matsushima T, Takahashi K. Serum pepsinogens as a screening test of extensive chronic gastritis. Gastroenterol Jpn 1987;22:133-141.
[51] Samloff I M, Varis K, Ihamaki T, Siurala M, Rotter J I. Relationships among serum pepsinogen I, serum pepsinogen II, and gastric mucosal histology. A study in relatives of patients with pernicious anemia. Gastroenterology 1982;83:204-209.
[52] Korstanje A, den Hartog G, Biemond I, Lamers C B. The serological gastric biopsy: a non-endoscopical diagnostic approach in management of the dyspeptic patient: significance for primary care based on a survey of the literature. Scand J Gastroenterol Suppl 2002;236:22-26.
[53] Oksanen A, Sipponen P, Miettinen A, Sarna S, Rautelin H. Evaluation of bood tests to normal gastric mucosa. Scand J Gastroenterol 2000;35:791-795.
[54] Varis K, Sipponen P, Laxen F, Samloff I M, Huttunen J K, Taylor P R, and The Helsinki Gastritis Study Group. Implications of serum pepsinogen I in early endoscopic diagnosis of gastric cancer and dysplasia. Scand J Gastroenterol 2000;35:950-956.
[55] Miki K. Gastric cancer screening using the serum pepsinogen test method. Gastric Cancer 2006;9:245-253.
[56] Agréus L, Kuipers E J, Kupcinskas L, Malfertheiner P, Di Mario F, Leja M, Mahachai V, Yaron N, van Oijen M, Perez Perez G, Rugge M, Ronkainen J, Salaspuro M, Sipponen P, Sugano K, Sung J. Rationale in diagnosis and screening of atrophic gastritis with stomach-specific plasma biomarkers. Scand J Gastroenterol 2012;47:136-147.
[57] Malfertheiner P, Sipponen P, Naumann M, et al. Helicobacter pylori eradication has the potential to prevent gastric cancer: a state-of-the-art critique. Am J Gastroenterol 2005;100:2100-2015.
[58] Correa P, Fontham E T, Bravo J C, et al. Chemoprevention of gastric dysplasia: randomized trial of antioxidant supplements and anti-helicobacter pylori therapy. J Natl Cancer Inst 2000;92:1881-1888.
[59] Leung W K, Lin S R, Ching J Y, et al. Factors predicting progression of gastric intestinal metaplasia: results of a randomised trial on Helicobacter pylori eradication. Gut 2004;53:1244-1249.
[60] Wong B C, Lam S K, Wong W M, et al. Helicobacter pylori eradication to prevent gastric cancer in a high-risk region of China: a randomized controlled trial. JAMA 2004;291:187-194.
[61] Wang J, Xu L, Shi R, et al. Gastric atrophy and intestinal metaplasia before and after Helicobacter pylori eradication: a meta-analysis. Digestion 2011;83:253-260.
[62] Di Mario F, Rugge M, Ferrara F, Dal Bò N, Slongo T, Guarnieri G, Marcello R, Caroli A, Heras Salvat H, Rigoli R, Dei Tos A P, Scarpignato C. Recovery of gastric function after Helicobacter pylori eradication and Acetium administration: A 6 years study in atrophic gastritis subjects. Digestive Disease Week, Orlando, USA, May 18-21, 2013. Abstract.
[63] Di Mario F, Rugge M, Ferrara F, Slongo T, Dal Bo' N, Guarnieri G, Marcello R, Caroli A, Heras Salvat H, Rigoli R, Dei Tos A P, Scarpignato C. Recovery of gastric function after Helicobacter pylori eradication and Acetium administration: A 6 years study in atrophic gastritis subjects. 21^{st} UEG Week, Berlin, Germany, Oct. 12-16, 2013. Abstract.
[64] Sprince H, Parker C, Smith C J, Gonzales L. Protective action of ascorbic acid and sulfur compounds against acetaldehyde toxicity: implications in alcoholism and smoking. Agents Actions 1975;5:164-173.
[65] Linderborg K, Marvola T, Marvola M, Salaspuro M, Färkkilä M, Väkeväinen S. Reducing carcinogenic acetaldehyde exposure in the achlorhydric stomach with cysteine. Alcohol Clin Exp Res 2011;35(3): 516-522.
[66] Salaspuro V, Hietala J, Kaihovaara P, Pihlajarinne L, Marvola M, Salaspuro M. Removal of acetaldehyde from saliva by a slow-release buccal tablet of L-cysteine. Int J Cancer 2002;97:361-364.
[67] Salaspuro V J, Hietala J M, Marvola M L, Salaspuro M P. Eliminating carcinogenic acetaldehyde by cysteine from saliva during smoking. Cancer Epidemiol. Biomark. Prev. 2006;15:146-149.
[67b] Maejima R, Iijima K, Kaihovaara P, Hatta W, Koike T, Imatani A, Shimosegawa T, Salaspuro M. Effects of ALDH2 genotype, PPI treatment and L-cysteine on carcinogenic acetaldehyde in gastric juice and saliva after intra-gastric alcohol administration. PLoS One. 2015;10:e0120397
[67c] Hellström PM, Hendolin P, Kaihovaara P, Kronberg L, Meierjohann A, Millerhov A, Paloheimo L, Sundelin H, Syrjänen K, Webb D-L, Salaspuro M. Slow-release L-cysteine capsule prevents gastric mucosa exposure to carcinogenic acetaldehyde: results of a randomised single-blinded, cross-over study of Helicobacter-associated atrophic gastritis. Scand. J. Gastroenterol 2016;52:230-237.
[68] Hoffmann D, Hecht S. Advances in tobacco carcinogenesis. In: Cooper C S, Grover P, editors. Handbook of experimental pharmacology. Heidelberg: Springer Verlag; 1990; pp. 63-102.
[69] Hoffmann D, Hoffmann I. The changing cigarette, 1950-1995. J Toxicol Environ Health 1997; 50:307-364.
[70] Salaspuro M. Acetaldehyde as a common denominator and cumulative carcinogen in digestive tract cancers. Scand J Gastroenterol 2009;24:1-15.
[71] Salaspuro V, Salaspuro M. Synergistic effect of alcohol drinking and smoking on in vivo acetaldehyde concentration in saliva. Int J Cancer 2004;111:480-486.
[72] Secretan B, Straif K, Baan R, Grosse Y, El Ghissassi F, Bouvard V, Benbrahim-Tallaa L, Guha N, Freeman C, Galichet L, Cogliano V. WHO International Agency for Research on Cancer Monograph Working Group. A review of human carcinogens-Part E: tobacco, areca nut, alcohol, coal smoke, and salted fish. Lancet Oncol 2009;10(11):1033-1034.
[73]. Koivurova O P, Ukkola O, Koivikko M, Ebeling T, Yliaska I, Koskela R, Blomster T, Ala-Rämi A, Kettunen O P, Karttunen T J, Suovaniemi O, Syrjänen K. Screening of the patients with autoimmune thyroid disease (AITD) and type 1 diabetes mellitus (DM1) for atrophic gastritis (AG) by serological biomarker testing (GastroPanel®). EC Gastroenterol. Digest. Syst. 2020;7:181-195.
[74]. Kurilovich S A, Belkovets A V, Reshetnikov O V, Openko T G, Malyutina S K, Ragino Y I, Scherbakova L V, Leja M, Paloheimo L, Syrjänen K. Stomach-specific biomarkers (GastroPanel) can predict the development of gastric cancer in Caucasian population: A longitudinal nested case-control study in Siberia. Anticancer Res 2016; 36:247-254.
[75]. Tu H, Sun L, Dong X, Gong Y, Xu Q, Jing J, Bostick R M, Wu X, Yuan Y A. Serological biopsy using five stom-ach-specific circulating biomarkers for gastric cancer risk assessment: A multi-phase study. Am J Gastroenterol 2017; 112:704-715.
[76]. Syrjänen, K. A Panel of serum biomarkers (GastroPanel®) in non-invasive diagnosis of atrophic gastritis. Systematic review and meta-analysis. Anticancer Res 2016; 36:5133-5144.
[77]. Zagari R M, Rabitti S, Greenwood D C, Eusebi L H, Vestito A, Bazzoli F. Systematic review with meta-analysis: diag-nostic performance of the combination of pepsinogen, gastrin-17 and anti-Helicobacter pylori antibodies serum assays for the diagnosis of atrophic gastritis. Aliment Pharmacol Ther 2017; 1-11,
[78]. Fine J P, Gray R J. A proportional hazards model for the subdistribution of a competing risk. J. Am. Stat. Assoc. 1999; 94:496-507.
[79]. Putter H, Fiocco M, Geskus R B. Tutorial in biostatistics: Competing risks and multi-state models. Stat. Med.

1. An acetaldehyde-binding preparation for use in a method for improving the structure and physiological function of gastric mucosa among subjects with acid-free stomach and/or Helicobacter pylori-associated (HP+) or autoimmune type (HP−) atrophic gastritis, the method comprising at least the following steps:
screening or examining a symptomless subject or a subject having symptoms indicating an autoimmune disease, dyspepsia or reflux symptoms, by quantitatively measuring concentration(s) of a) pepsinogen I (PGI), pepsinogen II (PGII), gastrin-17 (G-17) and Helicobacter pylori antibody (HpAb) biomarkers, or b) pepsinogen I (PGI) biomarker, or c) PGI biomarker, PGII biomarker and PGI/PGII biomarker ratio, or d) G-17 biomarker, from a blood, serum or plasma sample obtained from said subject, and
comparing obtained value(s) to a pre-determined reference range(s) and/or cut-off value(s), characterized by administering 50-500 mg of L-cysteine containing slow-releasing preparation according to a pre-determined dosage regime to subjects indicative of acid-free stomach and/or atrophic gastritis based on said measurement and comparison.

2. The acetaldehyde-binding preparation for use according to claim 1, wherein the value(s) is/are indicative for atrophic gastritis, if the PGI concentration in said sample is close to the lower limit or below the reference range or cut-off value, PGI/PGII ratio is close to the lower limit or below the reference range or cut-off value and G-17 concentration is close to the upper limit or above the reference range.

3. The acetaldehyde-binding preparation for use according to claim 1, wherein the reference range for PGI value is 30-160 μg/l, for PGII 3-20 μg/l, for PGI/PGII ratio 3 or below 3, for G-17S (stimulated) value 5-30 pmol/l, for G-17B (fast) 2-10 pmol/l and the reference range for HpAb 0-30 EIU.

4. The acetaldehyde-binding preparation for use according to claim 1, wherein the typical cut-off values for the biomarkers are selected from the group consisting of: PGI 30 μg/l, PGI/PGII ratio 3, G-17S value 5 pmol/l, G-17B 2 pmol/l and HpAb 30 EIU.

5. The acetaldehyde-binding preparation for use according to claim 1, wherein the preferred dosage regime is three 50-500 mg, more preferably 50-300 mg and most suitably 100-200 mg of L-cysteine containing and slow-releasing capsules a day for a period of at least one month.

6. The acetaldehyde-binding preparation for use according to claim 1, wherein the L-cysteine is bound to a pharmaceutically acceptable carrier, which controls the releasing speed of the L-cysteine.

7. The acetaldehyde-binding preparation for use according to claim 6, wherein the pharmaceutically acceptable carrier comprises one or more substances selected from the group consisting of: chitosans, alginates, aluminium hydroxide, sodium hydrogen carbonate, sodium carboxymethyl cellulose, and sodium hydrogen carbonate.

8. The acetaldehyde-binding preparation for use according to claim 1, wherein the L-cysteine contained in one capsule is released into the stomach for 0.5 to 8 hours.

9. The acetaldehyde-binding preparation for use according to claim 1, wherein the L-cysteine amount released in the conditions of the stomach is 40-80 mg in one hour.

10. The acetaldehyde-binding preparation according to claim 1 for use in preventing atrophic gastritis and its progression into gastric cancer.
